(19) United States
(12) Patent Application Publication
(10) Pub. No.: US 2026/0275426 A1
(43) Pub. Date: Sep. 17, 2026

(54) METHODS OF CLASSIFYING AND TREATING INFLAMMATORY BOWEL DISEASE

(71) Applicant: Baylor College of Medicine, Houston, TX (US)

(72) Inventors: Tor Savidge, League City, TX (US); Sik Yu So, Houston, TX (US); Shyam Raj Badu, Houston, TX (US)

(73) Assignee: Baylor College of Medicine, Houston, TX (US)

(21) Appl. No.: 19/469,086

(22) PCT Filed: Mar. 26, 2024
(86) PCT No.: PCT/US2024/021476

§ 371 Date: Sep. 25, 2025

Related U.S. Application Data
(60) Provisional application No. 63/492,312, filed on Mar. 27, 2023.

(51) Int. Cl. C12Q 1/689; A61K 31/196; A61K 31/4164; A61K 31/4245; A61K 31/445; A61K 31/496; A61K 31/4985; A61K 31/519; A61K 31/52; A61K 31/7048; A61K 31/717; A61K 35/00; A61K 35/741; A61K 35/745; A61K 36/68; A61K 38/14; A61K 45/06; C07K 16/1282; C07K 16/24; C07K 16/28

(52) U.S. Cl. CPC C12Q 1/689; A61K 31/196; A61K 31/4164; A61K 31/4245; A61K 31/445; A61K 31/496; A61K 31/4985; A61K 31/519; A61K 31/52; A61K 31/7048; A61K 31/717; A61K 35/741; A61K 35/745; A61K 36/68; A61K 38/14; A61K 45/06; C07K 16/1282; C07K 16/241; C07K 16/244; C07K 16/2839; A61K 2035/115; C12Q 2600/118; C12Q 2600/158

(57) ABSTRACT

Embodiments of the disclosure encompass methods and compositions for treating and/or identifying subjects having Inflammatory Bowel Disease (IBD). In certain embodiments, methods include measuring taxa occurrence frequencies in at least one microbiome sample from a subject suspected or having or being at risk for having IBD when certain taxa are enriched in the microbiome and/or when certain taxa are deficient in the microbiome, and particularly upon classification of their microbiome based on a taxa enrichment profile. In certain embodiments, an individual is determined to be a suitable donor for fecal microbiota transplant or is determined not to be a suitable donor for FMT based on classification of the taxa profile of their microbiome.

### CROSS REFERENCE TO RELATED APPLICATIONS

This application claims the benefit of priority of U.S. Provisional Patent Application 63/492,312 filed on Mar. 27, 2023, the contents of which are hereby incorporated by reference in their entirety.

### STATEMENT REGARDING FEDERALLY SPONSORED RESEARCH OR DEVELOPMENT

This invention was made with government support under Grant Number(s) AI152999 and AI124290 awarded by the National Institutes of Health. The government has certain rights in the invention.

### TECHNICAL FIELD

This disclosure relates at least to the fields of gastroenterology, bacteriology, cell biology, physiology, molecular biology, bioinformatics, diagnostics, and medicine.

### BACKGROUND

Inflammatory Bowel Diseases (IBD) are common, costly, and cause substantial morbidity starting in childhood. Although their causes are largely unknown, IBD are chronic inflammatory disorders characterized by an excessive gastrointestinal (GI) immune response in a genetically susceptible host. The two main IBD subtypes are Crohn's disease (CD) and ulcerative colitis (UC). Therapeutics such as small molecules and more recently immunotherapies are used as front-line therapies but their cost, side effects, development of resistance, and limited efficacy highlight the need for improved understanding and management paradigms.

The present disclosure provides solutions to long-felt needs in the art at least of diagnosing and treating gut-related issues.

### SUMMARY

Given the risk for antimicrobial resistant (AMR)-pathogens causing life-threating infections and/or quality of life diminishment associated with improper diagnosis, successful infectious disease management is critically dependent on identifying the most susceptible patient and determining the appropriate therapeutic regimen to facilitate rapid clinical intervention. Although the value of precision diagnosis and treatment is well-recognized, neither the current analytical technology nor our understanding of microbiome feature associations are sufficiently well developed to initiate effective implementation.

The present disclosure is directed to methods and compositions that provide for accurate diagnosis and treatment of underlying microbiome dysbiosis in an individual. The methods can determine if an individual has or is at risk for having CDI, IBS, IBD UC, or IBD CD. Embodiments of the disclosure provide methods of identifying individuals that have CDI, IBS, IBD UC, or IBD CD (compared to age-matched or sex-matched individuals in the general population who are considered to have a non-dysbiosed microbiome) and identifying individuals that do not have CDI, IBS, IBD UC, or IBD CD (compared to the general population who are considered to have a non-dysbiosed microbiome).

Embodiments of the disclosure encompass methods of a variety of steps that utilize measuring microbiome taxa occurrence frequencies and classifying the microbiome from a subject as being from one of five different clusters, each having a unique taxanomic-enriched signature profile. In specific embodiments, the bacteria that are enriched are enriched relative to subjects in the general population who are considered to have a non-dysbiosed microbiome. The present disclosure provides for identification of a novel IBD-microbiome cluster that can predict disease risk and treatment outcome, thereby improving patient management.

Methods described herein can include treating an individual having diarrhea comprising: measuring for one or more taxonomical features from a biological sample from the individual; and providing a therapy upon classification of the microbiome into Cluster 1, 2, 3, 4, or 5. In particular embodiments, an individual whose microbiome is classified as Cluster 1 or 3 are administered an effective amount of one or more suitable therapies, such as fecal microbiota transplant (FMT), and/or other IBD-related therapies or administering antibiotics and/or antimicrobial treatment to the individual. In specific embodiments, individual whose microbiomes are categorized in Clusters 2, 4, and/or 5 may not be treated with FMT, antibiotics, and other IBD-related therapies.

When appropriate, methods can comprise antibiotics and/or antimicrobial treatment comprising at least one of the antibiotics selected from a small molecule antibiotic, an antibiotic derived from a natural product, a microbial composition, an antibody suitable for neutralizing pathogenic infections, a therapeutic, contact isolation, and any combination thereof.

When appropriate, methods can comprise antibiotics and/or antimicrobial treatments comprising at least one of vancomycin, fidaxomicin, and bezlotoxumab. Methods can comprise treatment with fidaxomicin, and optionally the treatment dosage is at least 200 mg twice daily for 10 days, the treatment is vancomycin, and optionally the treatment dosage is at least 125 mg four times per day for 10 days, and/or the treatment is bezlotoxumab.

Methods can comprise pathogenic diarrhea classification or non-pathogenic causative diarrhea classification (e.g., lactose intolerance, non-celiac gluten sensitivity, celiac disease, hyperthyroidism, bile acid diarrhea, a number of medications, irradiation, cytotoxics, clinical infectious diseases, including viral induced diarrhea, etc.) characterized by measuring taxa occurrence frequencies in a subject microbiome, including by classifying the subject microbiome into one of five Clusters, wherein each Cluster is associated with a signature taxa enrichment profile.

Methods can comprise measuring of one or more taxonomical features comprising at least one of analyzing one or more nucleic acids in the sample, analyzing one or more metabolites in the sample, and analyzing one or more proteins in the sample. Methods can comprise nucleic acid analysis, wherein the nucleic acid is analyzed by sequencing, polymerase chain reaction, isothermal amplification, bioinformatics, or any combination thereof. Methods can comprise 16S ribosomal RNA analysis. Methods can comprise metabolite analysis by mass spectrometry, ELISA, chromatography, or any combination thereof. Methods can comprise protein analysis by mass spectrometry, ELISA, chromatography, Western blotting, immunoprecipitation, immunoelectrophoresis, or any combination thereof.

Methods can comprise reducing the administration of antibiotics and/or antimicrobial treatments to the individual when the individual has a Cluster associated with individuals that do not have IBD (e.g., Clusters 2, 4, and/or 5).

Methods can comprise reducing the administration of microbial such as FMT and/or supplementary dietary therapy to the individual when the individual has a Cluster associated with individuals that do not have IBD (e.g., Clusters 2, 4, and/or 5).

Methods may comprise measuring taxa occurrence frequencies in a subject microbiome leading to or resulting in classification of the subject microbiome into one of five Clusters, each of which is associated with a particular taxa enrichment profile.

In some embodiments, methods, compositions, and/or kits described herein may comprise removal (e.g., removed from weighted consideration, removed from inclusion in a kit/composition, etc.) of one or more Unclassified and/or Not Available taxa from consideration as part of a taxa enrichment profile of a cluster. In some embodiments, one or more Unclassified and/or Not Available taxa are removed from cluster 1. In some embodiments, one or more Unclassified and/or Not Available taxa are removed from cluster 2. In some embodiments, one or more Unclassified and/or Not Available taxa are removed from cluster 3. In some embodiments, one or more Unclassified and/or Not Available taxa are removed from cluster 4. In some embodiments, one or more Unclassified and/or Not Available taxa are removed from cluster 5. In some embodiments, one or more Unclassified and/or Not Available taxa are removed from clusters 1, 2, 3, 4, and/or 5.

In some cases, a subject microbiome is identified as being associated with Cluster 1. Such a cluster may be enriched at least in, or only in, Bacteroides, Blautia, Lachnospiraceae, and unclassified taxa, in specific embodiments. In some embodiments, Cluster 1 has a taxa profile that comprises 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified_074, Not_Available_2, Prevotella, Parasutterella, and Clostridium. In specific embodiments, cluster 1 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa. In specific cases, a Cluster 1 microbiome comprises loss of metabolically active Faecalibacterium.

In some cases, a subject microbiome is identified as being associated with Cluster 2. Such a cluster may be enriched at least in, or only in, Bacteroides, Blautia, and Faecalibacterium taxa, in certain embodiments. In some embodiments, Cluster 2 has a taxa profile that comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037. In specific embodiments, cluster 2 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

In some embodiments, a subject microbiome is identified as being associated with Cluster 3. Such a cluster may be enriched at least in, or only in, Bacteroides, Blautia, Enterobacteriaceae, Streptococcus, and unclassified taxa. In some embodiments, Cluster 3 has a taxa profile that comprises 1 or more of the following listed bacterial taxa: Bacteroides, Blautia, Streptococcus, Unclassified_123, Unclassified_071, Unclassified_129, Veillonella, Lactobacillus, Erysipelatoclostridium, Unclassified_072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified_075, Flavonifractor, Unclassified 064, Roseburia, Dialister, Unclassified_132, Intestinibacter, Prevotella, Clostridium XVIII, Alistipes, Not_Available 2, Unclassified_083, Akkermansia, Haemophilus, Fusobacterium, Romboutsia, Ruminococcus2, Fusicatenibacter, and Clostridium sensu stricto. In specific embodiments, cluster 3 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

In some embodiments, a subject microbiome is identified as being associated with Cluster 4. Such a cluster may be enriched at least in, or only in, Blautia, Lachnospiraceae, unclassified taxa, Faecalibacterium, and Bifidobacterium. In some embodiments, Cluster 4 has a taxa profile that comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified_074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV. In some embodiments, cluster 4 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

In some embodiments, a subject microbiome is identified as being associated with Cluster 5. Such a cluster may be enriched at least in, or only in, Prevotella. In some embodiments, Cluster 5 has a taxa profile that comprises 1 or more of the following listed bacterial taxa: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available_2, Bilophila, Barnesiella, Dialister, Butyricimonas, Clostridium, Intestinibacter, Paraprevotella, Haemophilus, Clostridium IV, Phascolarctobacterium, and Flavonifractor. In some embodiments, cluster 5 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Methods of the disclosure may encompass selection of one or more fecal samples that are suitable for FMT, analyzing at least one donor fecal sample microbiome to determine taxa Cluster enrichment, and selecting fecal samples that are not enriched in taxa Cluster 1 and/or Cluster 3 as suitable for FMT. In specific embodiments, a donor fecal sample is suitable for FMT when enriched in Cluster 2, Cluster 4, and/or Cluster 5. A donor fecal sample may be suitable for FMT when enriched in Prevotella, Blautia, Faecalibacterium, Bifidobacteria, and/or Bacteroides. In specific aspects, a donor microbiome fecal sample is selected at the time of fecal sample obtainment, although the donor microbiome fecal sample may be selected after fecal sample processing. In specific embodiments, the donor microbiome fecal sample is selected prior to or following administration of the FMT to a subject. A donor microbiome fecal sample may be selected at least 1 week, 2 weeks, 3 weeks, 4 weeks, 2 months, 3 months, 4 months, 5 months, or 6 months after administration of the FMT to a subject.

Embodiments of the disclosure include methods of diagnosing IBD in a subject, comprising: a) measuring taxa occurrence frequencies in at least one microbiome sample from a subject, b) determining that the subject has or is at risk of developing IBD when the microbiome sample comprises enrichment in taxa Bacteroides, Blautia, and Lachnospiraceae, and depletion of taxa Faecalibacterium, Bifidobacteria, and/or Eubacteria. Embodiments of the methods may include measuring taxa occurrence frequencies in a subject microbiome and may further comprise classification of the subject microbiome into one of five Clusters, wherein each Cluster is associated with a taxa enrichment profile, and classification of the subject as having or is at risk of developing IBD. In some embodiments, the subject's microbiome is not enriched as in Cluster 2, Cluster 3, Cluster 4, and/or Cluster 5. In some aspects, the loss of metabolically active Faecalibacterium is associated with IBD and/or CDI. Enrichment in calprotectin may be associated with IBD and/or CDI. In particular embodiments, the microbiome sample or samples are grouped into one of five clusters. In specific embodiments, a dominant Blautia cluster is associated with healthy subjects.

In embodiments, there are methods of diagnosing IBD in a subject, comprising: a) measuring taxa occurrence frequencies in at least one microbiome sample from a subject suspected of having IBD or at risk for having IBD; b) classifying the subject microbiome into one of five Clusters, wherein each Cluster is associated with a taxa enrichment profile, wherein a subject having a microbiome associated with Cluster 1 or Cluster 3 is determined to have IBD or be at risk for having IBD, wherein (I) Cluster 1 is enriched at least in, or only in, Bacteroides, Blautia, Lachnospiraceae, and unclassified taxa; or Cluster 1 is enriched in 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified_074, Not_Available_2, Prevotella, Parasutterella, and Clostridium; and (II) Cluster 3 is enriched at least in, or only in, Bacteroides, Blautia, Enterobacteriaceae, Streptococcus, and unclassified taxa; or Cluster 3 is enriched in 1 or more of the following listed bacterial taxa: Bacteroides, Blautia, Streptococcus, Unclassified_123, Unclassified_071, Unclassified_129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified_075, Flavonifractor, Unclassified_064, Roseburia, Dialister, Unclassified_132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

Kits of the disclosure may comprise one or more bacteria listed herein and may be enriched for one or more bacteria disclosed herein. Such bacteria may or may not be formulated as an FMT composition in the kit. In specific embodiments, the kit does not comprise FMT comprising the taxa profile of Cluster 1 or 3. In some embodiments, the kit does comprise FMT comprising the taxa profile of Cluster 2, 4, and/or 5. The kit may or may not comprise therapies other than FMT, such as those described elsewhere herein.

The individual may be of any kind, and the methods may be performed before, during, or after the individual has diarrhea, and the diarrhea may or may not be chronic. The methods may be performed when the individual is in need of IBD therapy, antibiotics, and/or antimicrobials of any kind or when the individual has already had IBD therapy, antibiotics, and/or antimicrobials of any kind. The methods may be performed as routine medical practice for an individual. The methods may be performed as preventative medical practice for an individual. The methods may be performed for an individual having a family history of IBD.

In some embodiments, unaffected IBD relatives who have a similar genetic susceptibility to biological family member(s) with IBD have a healthy microbiome composition. In specific embodiments, if the microbiome of the unaffected IBD relative changes to an IBD-associated microbiome cluster, these subjects will transition into high risk of developing active IBD or will have IBD. In specific embodiments, the microbiome of an unaffected IBD relative is screened periodically to ascertain the associated cluster, and if their microbiome develops as Cluster 1 or 3, appropriate therapeutic intervention may occur regardless of whether or not one or more symptoms of IBD occur.

In some embodiments, an FMT composition of any kind is seeded with Fecalisbacteria, Bifidobacteria, and/or Eubacteria (e.g. E. rectale), as these are all oxygen sensitive, and viability may be lost during FMT preparation and/or treatment procedures.

Certain embodiments of the present disclosure and associated inventions can be characterized through the following enumerated Aspects.

Aspect 1 is a method of treating and optionally diagnosing Inflammatory Bowel Disease (IBD) in a subject, comprising: a) measuring taxa occurrence frequencies in at least one microbiome sample from a subject, b) determining that the subject has or is at risk of developing IBD when the microbiome sample comprises enrichment in taxa Bacteroides, Blautia, and/or Lachnospiraceae, and depletion of taxa Faecalibacterium, Bifidobacteria, and/or Eubacteria, and c) administering a therapy for the IBD.

Aspect 2 is the method of aspect 1, wherein the therapy comprises fecal microbiota transplant (FMT), one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

Aspect 3 is the method of aspect 1, wherein the therapy comprises FMT.

Aspect 4 is the method of any one of aspects 1-3, wherein measuring taxa occurrence frequencies in a subject microbiome further comprises classification of the subject microbiome into one of five Clusters, wherein each Cluster is associated with a taxa enrichment profile.

Aspect 5 is the method of aspect 4, wherein: a) Cluster 1 is enriched at least in, or only in, Bacteroides, Blautia, Lachnospiraceae, and unclassified taxa; b) Cluster 2 is enriched at least in, or only in, Bacteroides, Blautia, and Faecalibacterium taxa; c) Cluster 3 is enriched at least in, or only in, Bacteroides, Blautia, Enterobacteriaceae, Streptococcus, and unclassified taxa; d) Cluster 4 is enriched at least in, or only in, Blautia, Lachnospiraceae, unclassified taxa, Faecalibacterium, Eubacteria, and Bifidobacterium; or e) Cluster 5 is enriched at least in, or only in, Prevotella.

Aspect 6 is the method of aspect 5, wherein Cluster 1 determines that the subject has or is at risk of developing IBD.

Aspect 7 is the method of aspect 5, wherein the subject's microbiome is not enriched as in Cluster 2, Cluster 3, Cluster 4, and/or Cluster 5.

Aspect 8 is the method of aspect 2 or 3, wherein the FMT is not enriched for taxa associated with Cluster 1 and/or Cluster 3.

Aspect 9 is the method of any one of aspects 1-8, wherein the loss of metabolically active Faecalibacterium, Bifidobacterium, and/or Eubacterium is associated with IBD and/or CDI.

Aspect 10 is the method of any one of aspects 1-9, wherein enrichment in calprotectin is associated with IBD and/or CDI.

Aspect 11 is the method of any one of aspects 1-10, wherein the microbiome sample or samples are grouped into one of five clusters.

Aspect 12 is the method of any one of aspects 1-11, wherein a dominant Blautia cluster is associated with healthy subjects.

Aspect 13 is the method of any one of aspects 2-12, wherein the FMT is enriched for taxa occurrences of Faecalibacterium, Eubacterium, and/or Bifidobacterium.

Aspect 14 is the method of any one of aspects 5-13, wherein cluster 1 taxa profile comprises 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified 074, Not_Available_2, Prevotella, Parasutterella, and Clostridium.

Aspect 15 is the method of aspect 14, wherein cluster 1 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 16 is the method of any one of aspects 5-13, wherein cluster 2 taxa profile comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available 2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037.

Aspect 17 is the method of aspect 16, wherein cluster 2 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 18 is the method of any one of aspects 5-13, wherein cluster 3 taxa profile comprises 1 or more of the following listed bacterial taxa: Bacteroides, Blautia, Streptococcus, Unclassified_123, Unclassified_071, Unclassified 129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified 075, Flavonifractor, Unclassified 064, Roseburia, Dialister, Unclassified 132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

Aspect 19 is the method of aspect 18, wherein cluster 3 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 20 is the method of any one of aspects 5-13, wherein cluster 4 taxa profile comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified 074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available 5, Ruthenibacterium, and Clostridium IV.

Aspect 21 is the method of aspect 20, wherein cluster 4 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 22 is the method of any one of aspects 5-13, wherein cluster 5 taxa profile comprises: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available_2, Bilophila, Barnesiella, Dialister, Butyricimonas, Clostridium, Intestinibacter, Paraprevotella, Haemophilus, Clostridium IV, Phascolarctobacterium, and Flavonifractor.

Aspect 23 is the method of aspect 22, wherein cluster 5 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 24 is the method for selecting fecal samples suitable for fecal microbiota transplant (FMT), comprising: analyzing at least one donor fecal sample microbiome to determine taxa Cluster enrichment, and selecting fecal samples that are not enriched in taxa Cluster 1 and/or Cluster 3 as suitable for FMT.

Aspect 25 is the method of aspect 24, wherein a donor fecal sample is suitable for FMT when enriched in Cluster 2, Cluster 4, and/or Cluster 5.

Aspect 26 is the method of aspect 24 or 25, wherein a donor fecal sample is suitable for FMT when enriched in Prevotella, Blautia, Faecalibacterium, Bifidobacteria, and/or Bacteroides.

Aspect 27 is the method of any one of aspects 24-26, wherein the donor microbiome fecal sample is selected at the time of fecal sample obtainment.

Aspect 28 is the method of any one of aspects 24-27, wherein the donor microbiome fecal sample is selected after fecal sample processing.

Aspect 29 is the method of any one of aspects 24-28, wherein the donor microbiome fecal sample is selected prior to administration of the FMT to a subject.

Aspect 30 is the method of any one of aspects 24-29, wherein the donor microbiome fecal sample is selected following administration of the FMT to a subject.

Aspect 31 is the method of any one of aspects 24-30, wherein the donor microbiome fecal sample is selected at least 1 week, 2 weeks, 3 weeks, 4 weeks, 2 months, 3 months, 4 months, 5 months, or 6 months after administration of the FMT to a subject.

Aspect 32 is the method of any one of aspects 24-31, wherein cluster 2 taxa enrichment comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available 2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037.

Aspect 33 is the method of aspect 32, wherein cluster 2 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 34 is the method of any one of aspects 24-31, wherein cluster 4 taxa enrichment comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified 074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available 5, Ruthenibacterium, and Clostridium IV.

Aspect 35 is the method of aspect 38, wherein cluster 4 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 40 is the method of any one of aspects 24-31, wherein cluster 5 taxa enrichment comprises 1 or more of the following listed bacterial taxa: Prevotella, Bacteroides, Faecalibacterium, Unclassified 071, Blautia, Roseburia, Unclassified 072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not Available 2, Bilophila, Barnesiella, Dialister, Butyricimonas, Clostridium, Intestinibacter, Paraprevotella, Haemophilus, Clostridium IV, Phascolarctobacterium, and Flavonifractor.

Aspect 36 is the method of aspect 40, wherein cluster 5 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 37 is a kit, comprising: (a) one or more bacteria selected from the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037; (b) one or more of the following listed bacteria: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified 074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV; and/or (c) one or more of the following listed bacteria: Prevotella, Bacteroides, Faecalibacterium, Unclassified_071, Blautia, Roseburia, Unclassified_072, Unclassified 075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not Available 2, Bilophila, Barnesiella, Dialister, Butyricimonas, Clostridium, Intestinibacter, Paraprevotella, Haemophilus, Clostridium IV, Phascolarctobacterium, and Flavonifractor.

Aspect 38 is the kit of aspect 37, wherein the kit comprises one or more species associated with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (a).

Aspect 39 is the kit of aspect 37 or 38, wherein the kit comprises one or more species associated with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (b).

Aspect 40 is the kit of any one of aspects 37-39, wherein the kit comprises one or more species associated with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (c).

Aspect 41 is the kit of any one of aspects 37-40, wherein one or more of the bacteria in (a) are formulated for FMT.

Aspect 42 is the kit of any one of aspects 37-41, wherein one or more of the bacteria in (b) are formulated for FMT.

Aspect 43 is the kit of any one of aspects 37-42, wherein one or more of the bacteria in (c) are formulated for FMT.

Aspect 44 is the kit of any one of aspects 37-43, wherein the kit further comprises one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

Aspect 45 is the kit of aspect 44, wherein the one or more anti-inflammatory drugs is an aminosalicylate.

Aspect 46 is the kit of aspect 45, wherein the aminosalicylate is mesalamine, balsalazide, and/or olsalazine.

Aspect 47 is the kit of any one of aspects 37-44, wherein the one or more immune system suppressors is azathioprine, mercaptopurine and/or methotrexate.

Aspect 48 is the kit of any one of aspects 37-44, wherein the one or more small molecules is tofacitinib, upadacitinib, and/or ozanimod.

Aspect 49 is the kit of any one of aspects 37-44, wherein the one or more biologics is infliximab, adalimumab, golimumab, certolizumab, vedolizumab, ustekinumab, and/or Risankizumab.

Aspect 50 is the kit of any one of aspects 37-44, wherein the one or more antibiotics is ciprofloxacin, metronidazole, vancomycin, fidaxomicin, and/or bezlotoxumab.

Aspect 51 is the kit of any one of aspects 37-44, wherein the anti-diarrheal medication is Psyllium powder, methylcellulose, and/or loperamide.

Aspect 52 is a method of diagnosing IBD in a subject, comprising: a) measuring taxa occurrence frequencies in at least one microbiome sample from a subject, b) determining that the subject has or is at risk of developing IBD when the microbiome sample comprises enrichment in taxa Bacteroides, Blautia, and/or Lachnospiraceae, and depletion of taxa Faecalibacterium, Bifidobacterium and/or Eubacterium.

Aspect 53 is the method of aspect 52, wherein measuring taxa occurrence frequencies in a subject microbiome further comprises classification of the subject microbiome into one of five Clusters, wherein each Cluster is associated with a taxa enrichment profile.

Aspect 54 is the method of aspect 53, wherein: a) Cluster 1 is enriched at least in, or only in, Bacteroides, Blautia, Lachnospiraceae, and unclassified taxa; b) Cluster 2 is enriched at least in, or only in, Bacteroides, Blautia, and Faecalibacterium taxa; c) Cluster 3 is enriched at least in, or only in, Bacteroides, Blautia, Enterobacteriaceae, Streptococcus, and unclassified taxa; d) Cluster 4 is enriched at least in, or only in, Blautia, Lachnospiraceae, unclassified taxa, Faecalibacterium, and Bifidobacterium; or e) Cluster 5 is enriched at least in, or only in, Prevotella.

Aspect 55 is the method of aspect 54, wherein Cluster 1 determines that the subject has or is at risk of developing IBD.

Aspect 56 is the method of aspect 54, wherein the subject's microbiome is not enriched as in Cluster 2, Cluster 3, and/or Cluster 5.

Aspect 57 is the method of any one of aspects 54-56, wherein the loss of metabolically active Faecalibacterium, Bifidobacterium, and/or Eubacterium is associated with IBD and/or CDI.

Aspect 58 is the method of any one of aspects 54-57, wherein enrichment in calprotectin is associated with IBD and/or CDI.

Aspect 59 is the method of any one of aspects 54-58, wherein the microbiome sample or samples are grouped into one of five clusters.

Aspect 60 is the method of any one of aspects 54-59, wherein a dominant Blautia cluster is associated with healthy subjects.

Aspect 61 is the method of any one of aspects 54-60, wherein cluster 1 taxa enrichment comprises 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified 074, Not_Available_2, Prevotella, Parasutterella, and Clostridium.

Aspect 62 is the method of aspect 61, wherein cluster 1 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 63 is the method of any one of aspects 54-62, wherein cluster 2 taxa enrichment comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available 2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037.

Aspect 64 is the method of aspect 63, wherein cluster 2 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 65 is the method of any one of aspects 54-64, wherein cluster 3 taxa enrichment comprises 1 or more of the following listed bacterial taxa: Bacteroides, Blautia, Streptococcus, Unclassified_123, Unclassified_071, Unclassified 129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified_075, Flavonifractor, Unclassified 064, Roseburia, Dialister, Unclassified 132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

Aspect 66 is the method of aspect 65, wherein cluster 3 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 674 is the method of any one of aspects 54-66, wherein cluster 4 taxa enrichment comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified 074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available 5, Ruthenibacterium, and Clostridium IV.

Aspect 68 is the method of aspect 67, wherein cluster 4 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacteria.

Aspect 69 is the method of any one of aspects 54-68, wherein cluster 5 taxa enrichment comprises 1 or more of the following listed bacterial taxa: Prevotella, Bacteroides, Faecalibacterium, Unclassified_071, Blautia, Roseburia, Unclassified 072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available 2, Bilophila, Barnesiella, Dialister, Butyricimonas, Clostridium, Intestinibacter, Paraprevotella, Haemophilus, Clostridium IV, Phascolarctobacterium, and Flavonifractor.

Aspect 70 is the method of aspect 69, wherein cluster 5 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 71 is the method of any one of aspects 54-70, further comprising administering one or more therapies to the subject.

Aspect 72 is the method of any one of aspects 54-71, further comprising administering one or more therapies to the subject identified as having a microbiome associated with Cluster 1 or Cluster 3.

Aspect 73 is the method of aspect 71 or 72, wherein the therapy comprises FMT, one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

Aspect 74 is the method of aspect 73, wherein the FMT is not enriched for taxa associated with Cluster 1 and/or Cluster 3.

Aspect 75 is the method of any one of aspects 73-74, wherein the FMT is not enriched for taxa occurrences of Faecalibacterium and/or Bifidobacterium.

Aspect 76 is a method of diagnosing IBD in a subject, comprising: a) measuring taxa occurrence frequencies in at least one microbiome sample from a subject suspected of having IBD or at risk for having IBD; b) classifying the subject microbiome into one of five Clusters, wherein each Cluster is associated with a taxa enrichment profile, wherein a subject having a microbiome associated with Cluster 1 or Cluster 3 is determined to have IBD or be at risk for having IBD, wherein (I) Cluster 1 is enriched at least in, or only in, Bacteroides, Blautia, Lachnospiraceae, and unclassified taxa; or Cluster 1 is enriched in 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified 074, Not_Available_2, Prevotella, Parasutterella, and Clostridium; and (II) Cluster 3 is enriched at least in, or only in, Bacteroides, Blautia, Enterobacteriaceae, Streptococcus, and unclassified taxa; or Cluster 3 is enriched in 1 or more of the following listed bacterial taxa: Bacteroides, Blautia, Streptococcus, Unclassified_123, Unclassified_071, Unclassified_129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified 075, Flavonifractor, Unclassified 064, Roseburia, Dialister, Unclassified 132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

Aspect 77 is the method of aspect 76, wherein cluster 1 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 78 is the method of aspect 76, wherein cluster 3 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

Aspect 79 is the method of any one of aspects 76-78, wherein the subject suspected of having IBD or at risk for having IBD has diarrhea or a family history of IBD.

Aspect 80 is the method of any one of aspects 76-78, wherein the subject suspected of having IBD or at risk for having IBD has a microbiome that comprises enrichment in taxa Bacteroides, Blautia, and/or Lachnospiraceae and/or has depletion of taxa Faecalibacterium, Bifidobacteria, and/or Eubacteria.

Aspect 81 is the method of aspect 79, wherein the diarrhea is chronic.

Aspect 82 is a method of diagnostic surveillance and prophylactic management of Inflammatory Bowel Disease (IBD) risk in a subject, comprising: a) measuring taxa occurrence frequencies in at least one microbiome sample from a subject, b) determining that the subject has or is at risk of developing IBD when the microbiome sample comprises enrichment in taxa Bacteroides, Blautia, and Lachnospiraceae, and depletion of taxa Faecalibacterium, Bifidobacteria, and Eubacteria, and c) administering a therapy for the IBD.

Aspect 83 is the method of aspect 82, wherein the subject has no diarrhea.

Aspect 84 is the method of aspect 82, wherein the subject has diarrhea.

Aspect 85 is the method of aspect 82, wherein the individual is healthy.

Aspect 86 is the method of any one of aspects 82-85, wherein the subject has a biological family member with IBD.

Aspect 87 is the method of any one of aspects 82-86, wherein the method is performed periodically.

Aspect 88 is the method of any one of aspects 82-87, wherein the method is performed annually.

Aspect 89 is the method of any one of aspects 82-88, wherein the subject has one or more IBD-gene linkage susceptibility loci.

Aspect 90 is the method of any one of aspects 82-89, wherein the subject has a microbiome Cluster 1 or Cluster 3 and/or has one or more IBD-gene linkage susceptibility loci.

Aspect 91 is the method of aspect 90, wherein the subject is provided an effective amount of prophylactic management of disease risk.

Aspect 92 is the method or kit of any one of the preceding aspects, wherein one or more Unclassified and/or Not Available taxa are removed from clusters 1, 2, 3, 4, and/or 5.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: Gut microbiome structure and composition in diarrheal patients and controls. (FIG. 1A) Beta-diversity plot shows that there are significant compositional shifts in disease groups, especially in patients with CDI and CD (ANOSIM p=0.001); (FIG. 1B) Family abundance plot showing compositional differences across disease groups.

FIG. 2: Compositional bias in human IBD. Weighted Jaccard abundance distance plots show expansion of Pathobiome (top) and Bacteroidaceae (bottom paget) in CD and UC patients compared with controls, with loss of for example Bifidobacteriaceae (bottom FIG. 2 Continued). The abundance of specific taxa is weighted (size of circle).

FIG. 3. Beta-diversity plot showing Enterbacteriaceae abundance as an example of the Pathobiome expansion that occurs in human IBD and CDI patients.

FIGS. 4A-4B. 16S rRNA sequencing based profiling of microbial community structure illustrated by Dirichlet multinomial mixtures (DMM) clustering. DMM clustering is a probability-based model in which the samples are categorized based on the frequency of the appearance of each taxa in that sample. In a comprehensive meta-analysis, DMM clustering is a method that classifies samples by cluster or enterotype associated with a specific disease type. (FIG. 4A) DMM identified 5 major clusters in our adult training 16S dataset. Top drivers include: Cluster 1—Bacteroides, Blautia, Lachnospiraceae; Other; Cluster 2 —Bacteroides, Blautia, Faecalibacterium; Cluster 3 —Bacteroides, Blautia, Enterobacteriaceae; Other, Streptococcus; Cluster 4 —Blautia, Lachnospiraceae; Other, Faecalibacterium, Bifidobacterium and Cluster 5 —Prevotella. (FIG. 4B) Microbiome cluster representation in different human diarrheal diseases.

FIG. 5. DMM clusters significantly differentiates different diarrheal disease specimens; Chi-squared analysis shows a significant cluster difference (Chi square p-value <2.2e-16) between disease groups in a combined analysis of 16S data using the Taxa4Meta profiler.

FIG. 6. Microbiome features associated with CDI patients (i.e. a high CDI risk score) are significantly associated with dysbiotic cluster 3.

FIG. 7. Top microbiome features that drive Cluster 1.

FIG. 8. Top microbiome features that drive Cluster 2.

FIG. 9. Top microbiome features that drive Cluster 3.

FIG. 10. Top microbiome features that drive Cluster 4.

FIG. 11. Top microbiome features that drive Cluster 5.

FIG. 12. Loss of Fecalibacteria in IBD-clusters 1 (and 3) demonstrated by 16S sequencing (left). Metaproteome analysis confirmed the cluster distribution of metabolically active Fecalibacteria in fecal samples of patients (right).

FIG. 13. Loss of Fecalibacteria in IBD and CDI patients. Shotgun metaproteome analysis confirmed the disease associated clustering by demonstrating loss of metabolically active Fecalibacteria in fecal samples of IBD and CDI patients.

FIG. 14. Clinical symptoms (PCDAI score) and fecal calprotectin levels are elevated in subjects with an IBD cluster.

FIG. 15. Independent cohort validation of fecal calprotectin2 abundance by cluster and IBD subtype.

FIG. 16. DIABIMNMUNE: longitudinal infant cohort. Early life human gut microbiome study-1 shows development of microbiome clusters modeled to the adult training set. Inter-enterotype transition probability at 1 and 3 years of age analyzed and visualized using the Markov chain-based approach. Transition probably matrix for 25-30M (prob<0.2 excluded) is provided on the right, where only transition probabilities greater than 0.2 are shown.

FIG. 17. TEDDY: longitudinal infant cohort. Early life human gut microbiome study-2 shows development of microbiome clusters modeled to the adult training set. Inter-enterotype transition probability at 1 and 3 years of age analyzed and visualized using the Markov chain-based approach. Transition probably matrix for 25-30M (prob<0.2 excluded) is provided on the right, where only transition probabilities greater than 0.2 are shown.

FIG. 18. RNASeq volcano plot (top) of genes expressed in blood specimens from age and sex matched TEDDY infants with a healthy versus IBD microbiome cluster. (Bottom) Summary of RNA SEQ data shows that transition from the IBD to healthy-cluster is associated with significant development of T cell differentiation and immune tolerance, which is lacking in IBD-associated cluster 1.

FIG. 19. Treatment/therapeutics, FMT considerations for treatment in IBD. FMT donor preparations reported in the literature demonstrate a microbiome community composition that is significantly different from healthy controls. Generally, FMT donor preparations lack key taxa present in the healthy gut microbiome cluster 4, notably Fecalibacteria and Bifidobacteria (Top). Donor preparations used to treat IBD patients are biased towards IBD risk clusters 1 and 3.

FIG. 20. Clinical FMT outcomes in UC patients stratified by donor cluster.

### DETAILED DESCRIPTION

Described herein are methods and compositions suitable for the treatment of disorders associated with dysbiosis of the microbiome. Use of the one or more compositions may be employed based on methods described herein. Methods and/or compositions described herein may be included as components of one or more kits suitable for treatment of disorders associated with dysbiosis of the microbiome. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

This application incorporates by reference herein in its entirety PCT Patent Application Serial. No. PCT/US23/64978, filed Mar. 27, 2023.

The use of the word “a” or “an” when used in conjunction with the term “comprising” in the claims and/or the specification may mean “one,” but it is also consistent with the meaning of “one or more,” “at least one,” and “one or more than one.”

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “Antimicrobial” as used herein is a general term for drugs, chemicals, or other substances that either kill or slow the growth of microbes. Among the antimicrobial agents are antibacterial drugs, antiviral agents, antifungal agents, and antiparasitic drugs. In patients this includes drugs and/or treatment that impacts microbiome community composition.

As used herein, the terms “arrays”, “microarrays”, and “DNA chips” refer to an array of distinct oligonucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. The oligonucleotides on the array may be designed to bind or hybridize to specific nucleic acids, such as a specific SNP or a specific CNV, for example.

The terms “Clostridioides difficile infection” “C. difficile infection” or “CDI” as used herein refers to an individual that has presence of Clostridioides difficile in their body to an extent and under conditions in which a sufficient level of toxins from the Clostridioides difficile results in diarrhea. This is in contrast to presence of Clostridioides difficile in an individual that is considered a carrier for the bacteria and that has no diarrhea.

The term “classifier” as used herein refers to an algorithm that implements a disease classification, notably CDI, IBS, IBD UC, and/or IBD CD diagnosis, or CDI, IBS, IBD UC, and/or IBD CD risk, or risk of C. difficile colonization. In other embodiments, the term refers to an algorithm that implements a disease classification for diagnosis or risk or risk of colonization for one or more pathogens other than C. difficile.

As used herein, the term “enterotype” refers to classification of living organisms (including mammals, such as primates, and including humans, for example) based on the bacteriological composition of their gut microbiota.

The term “feature” as used herein refers to a microbe, biological molecule, and/or metabolic pathway that is representative of a detectable difference between a control or reference standard and the corresponding microbe, biological molecule, and/or metabolic pathway in an individual with or at risk of developing CDI, IBS, IBD UC, and/or IBD CD. A feature may be the presence, absence, and/or levels of a microbe, nucleic acid sequence (such as 16S rRNA), protein, small molecule, metabolic pathway, and/or a combination thereof.

The term “pan-microbiome” as used herein refers to a composit of two or more microbiomes, for example, a composit of two or more data sets reflective of two or more microbiomes. A pan-microbiome may be larger than any single microbial community of an individual or a group. In some embodiments, a pan-microbiome includes two or more populations, two or more demographics, and/or data collected through two or more acquisition methodologies.

As used herein, the term “oligonucleotide” refers to a short chain of nucleic acids, either RNA, DNA, and/or PNA. The length of the oligonucleotide could be less than 10 base pairs, or at minimum or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs. The oligonucleotide can be synthesized using by methods including phosphodiester synthesis, phosphotriester synthesis, phosphite triester synthesis, phosphoramidite synthesis, solid support synthesis, in vitro transcription, or any other method known in the art.

As used herein, the term “PCR primer” refers to an oligonucleotide that is used to amplify a strand of nucleic acid in a polymerase chain reaction (PCR). Primers may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100% homology to the template the primers hybridize to, wherein the 3′ nucleotide of the primer is complementary to the template. In some embodiments, lower annealing temperatures are used for initial cycles, for example cycles 1, 2, 3, 4, and/or 5, of the reaction.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

“Subject” may refer to an organism that comprises a microbiome. In certain embodiments, it refers to a human patient. In certain embodiments, it refers to an animal.

Other objects, features and advantages of the present disclosure will become apparent from the following detailed description. It should be understood, however, that the detailed description and the specific examples, while indicating specific embodiments, are given by way of illustration only, since various changes and modifications within the spirit and scope of the immediate disclosure will become apparent to those skilled in the art from this detailed description

Targeted metagenomic sequencing is an emerging strategy to survey disease-specific microbiome biomarkers for clinical diagnosis and prognosis. However, this approach often yields inconsistent or conflicting results due to inadequate study power and experimental bias. To address this problem, the inventors developed Taxa4Meta, a bioinformatics pipeline explicitly designed to compensate for technical and demographic bias. The inventors designed and validated Taxa4Meta for accurate taxonomic profiling of 16S amplicon data acquired from different sequencing strategies. Taxa4Meta offers tremendous potential in identifying clinical dysbiotic features that can reliably predict human disease, validated comprehensively via re-analysis of individual patient 16S datasets. Specifically, Taxa4Meta facilitates pan-microbiome profiling of 16S features with excellent utility for stratification of IBD patients from diarrheal cases with Clostridioides difficile infection (CDI) and irritable bowel syndrome (IBS), who share common symptoms that are difficult to diagnose and manage. Thus, Taxa4Meta represents a novel approach to individual microbiome surveys to define dysbiosis at a population-scale level.

The new bioinformatics strategy was applied to develop microbiome biomarkers to assist in the clinical diagnosis and management of patients with IBD. A comprehensive re-analysis of >35,000 fecal 16S profiles generated across four continents covering the entire human life span (from infants to centenarians) was performed. This work enabled characterization of human gut microbiome development using pan-microbiome profiling as a strategy to minimize technological bias and identify core microbiome features. Using Taxa4Meta to investigate dysbiosis in IBD patients, the following significant discoveries were made: (1) in a detailed re-analysis of 4,491 IBD and matched control subjects from 36 clinical cohorts spanning North America, Europe and Australasia we identified an IBD-dominant microbiome cluster that is largely absent in healthy individuals and in patients with IBS or CDI serving as diarrheal disease controls; (2) the inventors demonstrated that this unique IBD-cluster can differentiate IBD from CDI and IBS patients, offering diagnostic utility; (3) the IBD-cluster is associated with microbiome immaturity associated with early childhood. Comprehensive RNAseq studies in matched blood specimens during infant development showed that the IBD-associated cluster is associated with immunological immaturity and lacks immune tolerance induced during normal microbiota development. Thus, the data show that IBD patients are associated with a gut microbiome immaturity that lack sufficient immune tolerance to microbial inflammatory triggers; (4) notably, by considering patient-donor microbiome cluster mismatching, the studies also indicate that Taxa4Meta can predict treatment outcomes in IBD patients who receive fecal microbiota transplants (FMT) and as such are a guide for precision microbiota therapy. The findings help disambiguate the role of the gut microbiota in the development of IBD during adolescence and adulthood. Moreover, these studies provide information for biomarker and therapeutic target discovery.

I. Diseases Associated with Microbiome Dysbiosis

The human gut microbiome comprises bacteria, viruses, and fungi ideally living symbiotically with their human host. Individual species and collective bacterial functions within the gut microbiome confer many benefits throughout life including metabolizing dietary contributions, educating the immune system, defending against pathogens, and contributing to overall health and optimal growth. The gut microbiome is affected by and influences pathologies including but not limited to inflammatory bowel disease (IBD; both ulcerative colitis (UC) and Crohn's disease (CD)), Clostridium difficile infection (CDI), and irritable bowel syndrome (IBS).

A. Clostridioides (Previously Termed Clostridium) difficile Infection (CDI)

Clostridioides difficile is a bacterium that causes an infection (CDI) of the large intestine (colon). Symptoms can range from diarrhea to life-threatening damage to the colon. The bacterium is often referred to as C. difficile or C. diff Illness from C. difficile typically occurs after use of antibiotic medications. It most commonly affects older adults in hospitals or in long-term care facilities. In the United States, about 200,000 people are infected annually with C. difficile in a hospital or care setting. Thankfully these numbers are trending lower than in previous years because of improved prevention measures. People not in care settings or hospitals also can develop C. difficile infection. Some strains of the bacterium in the general population may cause serious infections or are more likely to affect younger people. In the United States, about 170,000 infections occur annually outside of health care settings, and worryingly these numbers are increasing. Some people carry C. difficile bacteria in their intestines but never become sick. These individuals are carriers of the bacteria and may spread infections. Signs and symptoms usually develop within 5 to 10 days after starting a course of antibiotics. However, they may occur as soon as the first day or up to three months later. The most common signs and symptoms of mild to moderate C. difficile infection are: watery diarrhea three or more times a day for more than one day and/or mild abdominal cramping and tenderness. People who have a severe C. difficile infection tend to become dehydrated and may need to be hospitalized. C. difficile can cause the colon to become inflamed and sometimes form patches of raw tissue that can bleed or produce pus. Signs and symptoms of severe infection include: watery diarrhea as often as 10 to 15 times a day, abdominal cramping and pain (which may be severe), rapid heart rate, dehydration, fever, nausea, increased white blood cell count, kidney failure, loss of appetite, swollen abdomen, weight loss, and/or blood or pus in the stool. C. difficile infection that is severe and sudden, an uncommon condition, may also cause intestinal inflammation leading to enlargement of the colon (also called toxic megacolon) and sepsis. Sepsis is a life-threatening condition that occurs when the body's response to an infection damages its own tissues. People who have these conditions are generally admitted to an intensive care unit.

C. difficile bacteria enter the body through the mouth. They can begin reproducing in the small intestine. When they reach the large intestine (colon), they can release tissue-damaging toxins. These toxins destroy cells, produce patches of inflammatory cells and cellular debris, and cause watery diarrhea. When the bacteria are outside the colon, virtually anywhere in the environment, they are in a dormant state, or essentially quiescent. This enables them to survive for a long time in any number of places, including but not limited to human or animal feces, surfaces in a room, unwashed hands, soil, water, and/or food. When bacteria once again find their way into a person's digestive system, they begin to produce infection again. The ability of dormant C. difficile to survive outside the body enables the generally easy transmission of the bacterium, particularly in the absence of thorough hand-washing and cleaning.

Risk factors associated with developing a C. difficile infection include but are not limited to, taking antibiotics or other medications such as Clindamycin, Cephalosporins, Penicillin's, Fluoroquinolones, and/or potentially certain proton pump inhibitors. The majority of C. difficile infections occur in people who are or who have recently been in a health care setting, including hospitals, nursing homes and long-term care facilities, where germs spread easily, antibiotic use is common and people are especially vulnerable to infection. Additionally, certain medical conditions or procedures may increase an individual's susceptibility to a C. difficile infection, such as IBS, a weakened immune system from a medical condition or treatment (e.g., chemotherapy), chronic kidney disease, a gastrointestinal procedure, and/or other abdominal surgery. Additionally, age is a major risk factor for CDI infection.

Complications associated with C. difficile infection include but are not limited to: dehydration, kidney failure, toxic megacolon, bowel perforation, and/or death. The necessity for swift and correct diagnosis and initiation of appropriate therapeutic interventions are key steps in limiting the impact of this potentially devastating disease. Methods and compositions disclosed facilitate this process.

### B. Irritable Bowel Syndrome (IBS)

Irritable bowel syndrome (IBS) is a common disorder that affects the large intestine. Signs and symptoms include cramping, abdominal pain, bloating, gas, and diarrhea or constipation, or both. IBS is a chronic condition that will require long term management. Only a small number of people with IBS have severe signs and symptoms. Some people can control their symptoms by managing diet, lifestyle and stress. More-severe symptoms can be treated with medication and counseling.

The signs and symptoms of IBS vary but are usually present for a long time. The most common include: abdominal pain, cramping or bloating that is related to passing a bowel movement, changes in appearance of bowel movement, changes in how often you are having a bowel movement, and/or other symptoms that are often related include bloating, increased gas or mucus in the stool. Certain severe symptoms associated with IBS may include: weight loss, diarrhea at night, rectal bleeding, iron deficiency anemia, unexplained vomiting, difficulty swallowing, and/or persistent paint hat isn't relieved by passing gas or a bowel movement.

The precise cause of IBS is not yet known. But factors that appear to play a role in IBS disease progression include: muscle contractions of the intestine that are stronger and/or last longer than normal and/or weaker than normal, poor nervous system signaling, severe infection, early life stress, and/or changes in the gut microbiome. IBS symptom “flares” can be triggered by certain foods such as beverages, wheat, dairy, citrus fruits, beans, cabbage, milk and/or carbonated drinks, or stress.

Risk factors associated with IBS include being young, being female, having a family history of IBS, and/or having anxiety, depression and/or other mental health issues.

Major complications associated with IBS include chronic constipation or diarrhea that can cause hemorrhoids, a reduction in the quality of life, and exacerbation of mood disorders.

Correct diagnosis and management of IBS is an essential step in long term management of the disease. Methods and compositions disclosed facilitate this process.

### C. Inflammatory Bowel Disease (IBD)

Inflammatory bowel disease (IBD) is an umbrella term used to describe disorders that involve chronic inflammation of your digestive tract. The two major types of IBD include Ulcerative colitis (UC) which involves inflammation and development of ulcers along the superficial lining of the large intestine and rectum; and Crohn's disease (CD) which is characterized by inflammation of the lining of the digestive tract which can also involve the deeper layers of the digestive tract. Both ulcerative colitis and Crohn's disease usually are characterized by diarrhea, rectal bleeding, abdominal pain, fatigue and weight loss. IBD can be debilitating, and can sometimes lead to life-threatening complications.

Symptoms of IBD vary depending on the severity of the associated inflammation, and where in the digestive tract it occurs. Symptoms may range from mild to severe and may be interrupted by periods of remission. Symptoms common to both IBD UC and IBD CD include but are not limited to diarrhea, fatigue, abdominal pain and cramping, blood in the stool, reduced appetite, and/or unintended weight loss.

The exact causes of IBD remain elusive. However, it is thought that diet and stress may be involved, but perhaps these factors just aggravate the disease and are not the root cause. It is also thought that an immune system malfunction may also contribute to IBD development. When the immune system tries to fight off an invading virus or bacterium, an abnormal immune response causes the immune system to attack the cells in the digestive tract, too. Heredity also may play a role in that IBD is more common in people who have family members with the disease. However, most people with IBD don't have this family history.

Risk factors for development of IBD include but are not limited to age, race and/or ethnicity, family history, cigarette smoking and/or nonsteroidal anti-inflammatory medications (e.g., ibuprofen, naproxen sodium, etc.).

Complications associated with UC and/or CD include colon cancer, skin/eye/joint inflammation, medication side effects, primary sclerosing cholangitis, blood clots, bowel obstruction, malnutrition, fistulas, anal fissures, toxic megacolon, severe dehydration and/or perforation of the colon.

Correct diagnosis and management of IBD be it UC or CD is an essential step in long term management of the disease. Methods and compositions disclosed facilitate this process.

### II. Methods of Identifying Features, Evaluating Levels of Features, and Classifying Disease State

Disclosed herein are methods of identifying features, and evaluating the presence, absence, or levels of said features in a sample. In certain embodiments, a feature may also be described as a biomarker. In certain embodiments, one or more features are used to classify (e.g., diagnose) a disease state and/or identify one or more effective treatment options for a patient with an intestinal disorder characterized by microbiome dysbiosis (e.g., CDI, IBS, IBD UC, and/or IBD CD). In some embodiments, one or more features are used to diagnose a disease state and/or identify one or more effective treatment options for a patient with an intestinal disorder characterized by diarrhea.

In certain embodiments, a feature is a taxonomical classification. In certain embodiments, a feature is the presence, absence, or level of one or more microbial taxonomic units (e.g., genera, species, etc.). In certain embodiments, a feature is a metabolic pathway. In some embodiments, disclosed herein are methods of using a pan-microbiome profiling pipeline as a method suitable for identification of certain core features that can be used for accurate downstream diagnosis, accurate method of treatment prescription, and/or treatment composition determination.

It is contemplated that features may be evaluated based on one or more associated gene products. In some embodiments, a gene product is an amplicon complementary to at least a portion of a gene. In some embodiments, a gene product is an RNA transcript. In some embodiments, a gene product is a structural and/or functional RNA transcript. In some embodiments, a gene product is a protein expressed by an RNA transcript. In some embodiments, a gene product is a metabolic pathway associated with expression of a number of gene products. In some embodiments, a gene product is a metabolic pathway associated with expression of a number of gene products from a number of different species.

In some embodiments, features are identified using a pan-microbiome approach. In some embodiments, utilization of a pan-microbiome approach to identify features can reduce technical and/or demographic bias. In some embodiments, a pan-microbiome approach is a method that identifies and selects classifier features by analysis of microbiome data generated from two or more different sequencing strategies (e.g., 16S sequencing strategies) and/or two or more populations (e.g., two or more demographically distinct populations).

In certain embodiments, a meta-analysis to determine the presence, absence, levels, expression, and/or activity of one or more features disclosed herein for correlation with a disease state can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a marker gene or activity. Normalization of the expression of a marker gene is preferably achieved by dividing the expression level of the individual marker gene to be normalized by the respective individual median expression of this marker genes, wherein said median expression is preferably calculated from multiple measurements of the respective gene in a sufficiently large cohort of test individuals. In some embodiments, a test cohort comprises at least 3, 10, 100, 200, 1000 individuals or more including all values and ranges thereof. In some embodiments, dataset-specific bias can be removed or minimized allowing multiple datasets to be combined for meta-analyses (See Sims et al. BMC Medical Genomics (1:42), 1-14, 2008, which is incorporated herein by reference in its entirety). In some embodiments, a meta-analysis cohort comprises the combination of 3, 6, 9, 12, 15, 18, 21, 24, 27, 30, 33, 36, 39, 42, 45 test cohorts or more including all values and ranges thereof.

### A. Identification of Features Suitable for Classification of Disease State

In some embodiments, a step in determining features suitable for classification of microbiome dysbiosis associated disease state and/or determination of appropriate treatment methods comprises simulation of full-length and/or region-specific 16S amplicon data. In certain embodiments, simulation of full-length and/or region-specific 16S amplicon data can be based on reference data bases (e.g., NCBI 16S rRNA RefSeq database (downloaded in July 2019), Ribosomal Database Project (RDP) database (release 11.5) (Cole et al., 2014), etc.). In certain embodiments, bioinformatics tools such as cutadapt (version 2.4)(Martin, 2011)d can be used to extract sequence fragments as full-length amplicons of targeted 16S variable regions (V1-V3, V3-V5, V4 and V6-V9) based on the forward and reverse primers (e.g., primers as listed in Table 22). In some embodiments, an error rate is permitted during sequence extraction, for example, an error rate of 0.05, 0.1, 0.15, 0.2, 0.25, etc. In certain embodiments, an error rate of 0.2 is permitted during sequence extraction. In certain embodiments, sequence length trimming and/or random simulation of sequence abundance and quality scores are performed for specific benchmarking purposes.

In some embodiments, a step in determining features suitable for classification of microbiome dysbiosis associated disease state and/or determination of appropriate treatment methods comprises benchmarking of sequence clustering and denoising using simulated amplicons, optionally with variable length. In certain embodiments, when benchmarking the accuracy of clustering or denoising for amplicon data using variable sequence lengths, random count ranging from 1 to 50 (e.g., 1, 2, 3, 4, 5 . . . 45, 46, 47, 48, 49, or 50) can be assigned for one or more or all parent full-length amplicons extracted from a reference database (e.g., NCBI 16S rRNA RefSeq sequences). In some embodiments, sequencing data may be generated in the reverse orientation and/or the forward orientation. In some embodiments, traditional 454 data is generated from reverse orientation, and length trimming from either forward or reverse orientation is applied to one or more, or each type of amplicon data. In some embodiments, length trimming results in 100, 150, 170, 200, 250, 300, 350, 400 and/or 450 bases for variable regions, e.g., V1-V3, V3-V5 and V6-V9 amplicon data. In some embodiments, length trimming results in 100, 150, 170, 200 and/or 250 bases for variable regions, e.g., V4 amplicon data. In some embodiments, random phred quality score (ASCII_BASE=33) ranging from 30 to 42 can be assigned to each base for sequencing denoising. In some embodiments, simulated amplicons of each sequence length represents one sample. In some embodiments, one or more or all samples with the same sequence orientation from the same 16S region can then be included for closed-reference or de novo clustering (e.g., using UCLUST (v1.2.22) Edgar, 2010) or VSEARCH (v2.9) (Rognes et al., 2016) or denoising using DADA2 (v1.8) (Callahan et al., 2016)). In some embodiments, sequence similarity thresholds including 0.97, 0.99 and 1.00 can be evaluated for each clustering strategy. In some embodiments, databases (e.g., the SILVA database (release 132)) can be used for closed-reference OTU picking. In some embodiments, simulated amplicons of variable length originating from the same parent full-length amplicon have the same sequence counts, in such situations, pairwise Spearman correlation analysis can be performed for sequence counts of any two sequence lengths (as two independent samples) in one or more OTU count tables

In some embodiments, a step in determining features suitable for classification of microbiome dysbiosis associated disease state and/or determination of appropriate treatment methods comprises benchmarking of taxonomic over-classification. In some embodiments, taxonomic over-classification for short amplicon data represents an important criteria for controlling false positives. In some embodiments, using default parameters in the Bayesian-based Lowest Common Ancestor (BLCA) tool (Gao et al., 2017) and its default database of NCBI 16S rRNA RefSeq can be used to annotate random and repeat sequences that were previously generated for benchmarking IDTAXA and other annotation tools (Murali et al., 2018). In some embodiments, full-length 16S amplicons of unannotated sequences (e.g., at least down to family rank) are extracted from a suitable database (e.g., RDP database (release 11.5)) and are used for testing BLCA. In certain embodiments, BLASTN search of unannotated sequences against a suitable reference database (e.g., NCBI 16S rRNA RefSeq database) can be used to confirm that no best hits are identified at 97% threshold applied to either or both sequence identity and coverage. In some embodiments, simulated amplicons of unannotated RDP sequences are tested using different thresholds of sequence coverage and identity (e.g., ranging from 0.85 to 1.00 in BLCA). In some embodiments, 5, 6, 7, 8, 9, 10, 11, 12, 13, 14, 15, or more iterations of random sub-sampling (e.g., 1%, 2%, 3%, 4%, 5%) and BLCA annotation on those unannotated amplicons are performed for statistical determination of optimal sequence coverage and identity required for BLCA. In some embodiments, ten iterations of random sub-sampling (1%) and BLCA annotation on those unannotated amplicons are performed for statistical determination of optimal sequence coverage and identity required for BLCA. In some embodiments, taxonomic over-classification rate is defined as the classifiable proportion of unannotated amplicons at species level. In some embodiments, the confidence score of taxonomic assignment is not considered at this stage.

In some embodiments, a step in determining features suitable for classification of microbiome dysbiosis associated disease state and/or determination of appropriate treatment methods comprises benchmarking of taxonomic accuracy using simulated amplicons of variable length. In some embodiments, benchmarking taxonomic accuracy of BLCA, simulated amplicons of variable length are generated by trimming full-length amplicons derived from a suitable database (e.g., NCBI 16S RefSeq) from either forward or reverse orientation. In some embodiments, trimming of full-length amplicons results in 80, 100, 120, 140, 160, 180, 200, 220, 240, 260, 280, 300, 320, 340, 360, 380, 400, 420, 440, and/or 460 bases. In some embodiments, trimming of full-length amplicons results in 100, 150, 170, 200, 250, 300, 350, 400 and 450 bases for V1-V3, V3-V5 and V6-V9 amplicon data, and 100, 150, 170, 200 and 250 bases for V4 amplicon data. In some embodiments, in addition to the known taxonomic lineage, the parent 16S sequences of simulated amplicons are also present in the BLCA default reference database using NCBI 16S RefSeq, thus taxonomic misclassification can be evaluated. In some embodiments, misclassification rate is defined as the proportion of incorrect annotations for simulated amplicons. In some embodiments, to further determine the optimal confidence threshold of BLCA for mitigating misclassification, amplicons with a selected sequence length range are combined to calculate the proportion of correct versus incorrect annotations using defined thresholds. In some embodiments, the already known taxonomic lineage, true positive (TP) and false negative (FN) hits are correct annotations, whereas true negative (TN) and false positive (FP) hits are incorrect annotations.

In most embodiments, a step in determining features suitable for classification of microbiome dysbiosis associated disease state and/or determination of appropriate treatment methods comprises design of a data analysis pipeline. In certain embodiments, the data analysis pipeline is the Taxa4Meta pipeline. In certain embodiments, data analysis pipelines are generated as a function of benchmarking results. In certain embodiments, a new computational pipeline “Taxa4Meta” can be used to analyze 16S amplicon data with an optimal range of variable sequence lengths. In some embodiments, such a pipeline implements several open-source programs, such as VSEARCH (Rognes et al., 2016) for stringent clustering with a known identity range (e.g., 90%, 91%, 92%, 93%, 94%, 95%, 96%, 97%, 98%, 99%, 100% identity; preferably 99% identity). In some embodiments, open-source programs such as VSEARCH can be optimized for 16S amplicon data with the selected variable lengths after quality trimming. In some embodiments, BLCA (Gao et al., 2017) can be used with optimal region-specific confidence thresholds for stringent species annotation of OTUs. In certain embodiments, IDTAXA (Murali et al., 2018) can be utilized for annotating OTUs that cannot be annotated down to species resolution. In certain embodiments, collapsed taxonomic profiles from OTU tables are used for downstream analyses during 16S meta-analysis.

In some embodiments, a step in determining features suitable for classification of microbiome dysbiosis associated disease state and/or determination of appropriate treatment methods comprises benchmarking of taxonomic profiling accuracy comparing new data analysis pipelines (e.g., Taxa4Meta) with other standard 16S data analysis pipelines. In some embodiments, the feasibility and/or accuracy of different 16S pipelines are tested using the simulated and experimental datasets (McIntyre et al., 2017; Saulnier et al., 2011), and optionally the tests are designed to retain reads for accurate sequence clustering and for improved taxonomic accuracy. In some embodiments, simulated datasets are prepared from a suitable data base (e.g., NCBI 16S RefSeq as indicated above). In some embodiments, full-length amplicons of V1-V3, V3-V5, V4 and V6-V9 are simulated, and random sequence count ranging from 1 to 50 and random phred quality score (ASCII_BASE=33) ranging from 30 to 42 are generated for each full-length amplicon. In some embodiments, further length trimming is performed for one or more or each of the full-length amplicons, for example but not limited to, V1-V3 forward amplicons (200, 250, 300, 350, 400 and 450 bases), V1-V3 reverse amplicons (300, 350, 400 and 450 bases), V3-V5 forward amplicons (250, 300, 350, 400 and 450 bases), V3-V5 reverse amplicons (300, 350, 400 and 450 bases), both forward and reverse amplicons of V4 (200 and 250 bases), V6-V9 forward amplicons (300, 350, 400 and 450 bases), V6-V9 reverse amplicons (250, 300, 350, 400 and 450 bases). In some embodiments, trimmed amplicons from the same sequence orientation of the same 16S variable region are combined for benchmarking different 16S pipelines. In some embodiments, NCBI 16S taxonomic lineage of NCBI 16S RefSeq is used as the ground truth (reference annotations) for comparison. In some embodiments, a cohort (e.g., a Korean stool microbiome dataset (Whon et al., 2018) with the same DNA extracts used for 454 V1-V4, Illumina V1-V3, Illumina V3-V4, Illumina V4, and Illumina shotgun metagenomic sequencing is used as the real human microbiome dataset for benchmarking different 16S pipelines. In some embodiments, primers retained in the sequence reads are removed by positional trimming. In some embodiments, Illumina paired-end reads are merged (e.g., using USEARCH (version 8.1.1831)) with certain parameters (e.g., default parameters) prior to benchmarking 16S pipelines. In certain embodiments, key 16S analysis pipelines can include DADA2-IDTAXA, DADA2-RDP, UCLUST-UCLUST, USEARCH-RDP, Taxa4Meta, Kraken2 and/or MetaPhlAn2. In some embodiments, key 16S analysis pipelines DADA2-IDTAXA, DADA2-RDP, UCLUST-UCLUST, USEARCH-RDP, Taxa4Meta, Kraken2 and/or MetaPhlAn2 are benchmarked with simulated amplicons and/or ground truth datasets (e.g., Korean human microbiome dataset).

In some embodiments, an analysis procedure for a DADA2-IDTAXA pipeline can be performed. In some embodiments, DADA2 (version 1.8) is used for denoising amplicon data after quality filtering with a maximum expected error (e.g., of 2) and a minimum base length (e.g., of 200 bases). In some embodiments, IDTAXA together with its pre-built RDP training set (version 16) is used for taxonomic annotation with the confidence threshold (e.g., of 70) using a number of bootstraps (e.g., 100 bootstraps). In some embodiments, IDTAXA based analysis can only go down to genus level. In some embodiments, an analysis procedure for DADA2-RDP pipeline can be performed. In some embodiments, DADA2 (version 1.8) is used for denoising amplicon data after quality filtering with a maximum expected error (e.g., of 2) and minimum base length (e.g., of 200 bases). In some embodiments, RDP Naive Bayesian Classifier algorithm implemented in DADA2's assignTaxonomy function together with its pre-formatted RDP training set (version 16) is used for taxonomic annotation using a minimum bootstrap confidence (e.g., a minimum bootstrap confidence of 50). In some embodiments, a DADA2-RDP analysis can go down to species level. In some embodiments, an analysis procedure for a UCLUST-UCLUST pipeline can be performed. In some embodiments, UCLUST (version 1.2.22q) is used for clustering amplicon data with known sequence similarity (e.g., of 97%) after quality filtering with the minimum quality threshold (e.g., of 20) and a minimum base length (e.g., of 140 bases). In some embodiments, representative sequence(s) of OTUs are selected (e.g., with pick rep_set.py script) with default parameters. In some embodiments, UCLUST implemented in assign_taxonomy.py script together with SILVA database (release 123; choice of silva_132_97_16S.fna) is used for taxonomic annotation, which can be down to species level using a minimum bootstrap confidence (e.g., of 0.5). In some embodiments, one or more or all procedures are completed in the QIIME platform (version 1.9.1). In some embodiments, such a pipeline is similar to the meta-analysis method used by Mancabelli et al. (2017). In some embodiments, an analysis procedure for USEARCH-RDP pipeline can be performed. In some embodiments, USEARCH is used for clustering amplicon data with known sequence similarity (e.g., 100% sequence similarity) after quality filtering with a maximum expected error (e.g., of 2) and a minimum base length (e.g., of 200 bases). In some embodiments, RDP classifier (version 2.12) together with RDP training set (version 16) is used for taxonomic annotation, which can be down to species level using a minimum bootstrap confidence (e.g., of 0.5). In some embodiments, such a pipeline is similar to the meta-analysis method used by Duvallet et al. (2017)). In some embodiments, an analysis procedure for the Taxa4meta pipeline can be performed. In some embodiments, Taxa4Meta (e.g., version 1.22) is used for clustering amplicon data after quality filtering with a maximum expected error (e.g., of 2) and a selected range of variable lengths, optionally as suggested by Taxa4Meta itself. In some embodiments, taxonomic annotation by Taxa4Meta binary classifier can be down to species level. In some embodiments, an analysis procedure for Metagenomic classifiers can be performed. In some embodiments, Paired-end sequences are trimmed and filtered to meet a maximum expected error (e.g., of 2) with a minimum read length (e.g., of 50). In some embodiments, Kraken2 (version 2.0.8) with its pre-built database (minikraken2_v2_8 GB_201904_UPDATE) with default parameters is used for taxonomic profiling for shotgun metagenomic data. In some embodiments, MetaPhlAn2 (version 2.7.7) with it default database (mpa_v20_m200) with default parameters is used for taxonomic profiling for shotgun metagenomic data. In some embodiments, Kraken2 family-level abundance results are used as the reference for comparisons across different 16S pipelines. In some embodiments, given the high precision on species identification, MetaPhlAn2 species-level abundance results are used as the reference for evaluating species calls of different 16S pipelines. In some embodiments, a pseudo sample is created by averaging each family-level abundance of all WGS samples (e.g., 27 WGS samples), then the abundance-weighted Jaccard distance is calculated between the pseudo sample and any real sample analyzed by different pipelines.

In some embodiments, a step in determining features suitable for classification of microbiome dysbiosis associated disease state and/or determination of appropriate treatment methods comprises microbiome meta-analysis of diarrheal microbiome datasets. In some embodiments, one or more diarrheal datasets are run on the Taxa4Meta pipeline adopted optimal taxonomic thresholds for each 16S variable region. In some embodiments, relative abundance of collapsed species profiles generated from Taxa4Meta OTU count tables are used with or without rarefaction. In some embodiments, relative abundance of collapsed species profiles generated from Taxa4Meta OTU count tables require a minimum number of reads per sample. In some embodiments, a minimum number of reads per sample is 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500 or more or any range derivable therein. In some embodiments, a minimum number of reads per sample is 1,000 reads per sample. In some embodiments, if species is assigned by Taxa4Meta-BLCA, the taxonomic lineage from NCBI 16S RefSeq is adopted for that species to avoid inconsistency in taxonomic lineage. In some embodiments, merging of Taxa4Meta collapsed species of is based on taxonomic lineages.

In some embodiments, a step in determining features suitable for classification of microbiome dysbiosis associated disease state and/or determination of appropriate treatment methods comprises determining predictive metagenome functions. In some embodiments, predictive metagenome functions can be determined using open source software, e.g., PICRUSt2 (see e.g., Holmes et al., Generative models for microbial metagenomics. PLoS One 7, (2012)). In some embodiments, default Taxa4Meta parameters, OTU count tables, and/or OTU sequences are used to infer metabolic pathway abundance profiles for one or more datasets. In some embodiments, merging of PICRUSt2 pathway profiles is based on MetaCyc pathway IDs. In some embodiments, either or both LEfSe analysis (one-against-one test mode; version 1.0) and random forest (RF)-based feature ranking (default parameters in Orange version 3.20) are performed using pathway abundance profiles for diseased (e.g., CDI, IBD CD, IBD UC, and/or IBS) and/or control subjects. In some embodiments, mean decrease accuracy (MDA) score from RF-based analysis is used to rank pathways. In some embodiments, the top 20 pathways must be listed by both RF-based feature ranking result and LEfSe analysis result. In some embodiments, the top 2, 5, 10, 15, 20, 25, 30, 35, 40, 45, 50, 55, 60, 65, 70, 75, 80, 85, or 90 pathways are listed by both RF-based feature ranking results and LEFSe analysis results. In some embodiments, the top ranked pathways are selected for subsequent analysis. In some embodiments, the top ranked pathways are the top 2, 5, 10, 15, 20, 25, 30, 35, 40, 45, 50, 55, 60, 65, 70, 75, 80, 85, or 90 pathways or any range derivable therein. In some embodiments, the top ranked pathways are features indicative of a disease state and/or suitable for binary classification of disease state.

In some embodiments, data (e.g., relative abundances, associations, metabolic pathways, etc.) associated with a collection of one or more OTUs is collaposed into an enterotype. In some embodiments, an enterotype encompasses two or more OTUs. In some embodiments, an OTU may be collapsed into a simplified genera designation. In some embodiments, an OTU is not collapsed into a simplified genera designation.

In some embodiments, a step in determining features suitable for classification of microbiome dysbiosis associated disease state and/or determination of appropriate treatment methods comprises determining α-Diversity and/or β-diversity. In some embodiments, one or more α-diversity indices are calculated at OTU levels. In some embodiments, α-diversity indices are the Shannon index (e.g., alpha_diversity.py in QIIME v1.9.1) and/or the richness index (e.g., breakaway package version 4.7.5). In some embodiments, QIIME v1.9.1, principal coordinate analysis (PCoA) with abundance-weighted Jaccard distance metric is applied for β-diversity analysis using combined collapsed species profile. In some embodiments, ANOSIM test for group comparison is performed using the beta-diversity distance profile and the permutations of 999.

In some embodiments, a step in determining features suitable for classification of microbiome dysbiosis associated disease state and/or determination of appropriate treatment methods comprises fitting factors onto β-diversity ordination plot. In some embodiments, fitting factors (e.g., taxa) onto a two-dimensional ordination plot (e.g., first two coordinates) is performed using the envfit function in vegan package (version 2.5-7) or a suitable alternative program. In some embodiments, taxonomic abundance profile at family level is used as one of or the only factor in this analysis. In some embodiments, significance of fitted factors is established using the permutation of 999 in the envfit run.

In some embodiments, a step in determining features suitable for classification of microbiome dysbiosis associated disease state and/or determination of appropriate treatment methods comprises microbiome enterotyping. In some embodiments, microbiome enterotyping is performed with family abundance profiles of one or more or all meta-analysis training sets. In some embodiments, Dirichlet multinomial mixtures (DMM) algorithm, a classical method for clustering community profile data, is used for microbiome enterotyping.

In some embodiments, a step in determining features suitable for classification of microbiome dysbiosis associated disease state and/or determination of appropriate treatment methods comprises supervised classification and/or independent cohort validation. In some embodiments, supervised classification procedures are performed using Orange software (Demšar et al., 2013) (e.g., version 3.20) or a suitable alternative thereof, and applied to the reported cohorts with clinical definitions. In some embodiments, an original sample grouping information from each cohort is adopted. In some embodiments, such an adoption is done so the gold standard definition is clear for each sample. In some embodiments, random forest-based feature ranking was used as a first pass to select the top 100 input features (e.g., taxa, or biochemical pathways) for downstream supervised learning. In some embodiments, unless performing sub-sampling, input samples are used for training procedure. In some embodiments, supervised classification is performed using individual learning algorithms including but not limited to Random Forest (RF), Support Vector Machine (SVM), Naïve Bayes (NB), and/or Neural Network (NN). In some embodiments, a Stack model as an aggregated meta-learner of RF, SVM and NB is assessed. In some embodiments, a 5-fold cross-validation method is applied for sub-sampling of training and test data during a training procedure. In some embodiments, receiver-operating-characteristic (ROC) analysis is performed using the training results. In some embodiments, values of area-under-the curve (AUC) and classification accuracy (CA) are calculated to evaluate the performance of each classification model. In some embodiments, a suitable AUC value is more than 0.80, 0.81. 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 or any range derivable therein. In some embodiments, a preferred AUC value is more than 0.95, 0.96, 0.97, 0.98, or 0.99 or any range derivable therein. In some embodiments, CA refers to the proportion of correct predicted samples from the classification model compared to the original clinical diagnosis. In some embodiments, a suitable CA value is more than 0.80, 0.81. 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 or any range derivable therein. In some embodiments, a preferred CA value is more than 0.90, 0.91, 0.92, 0.93, 0.94, 0.95, 0.96, 0.97, 0.98, or 0.99 or any range derivable therein. In some embodiments, independent validation of classification models is performed using datasets of recently reported microbiome surveys of human diarrheal diseases that were not included in the training set. In some embodiments, one or more validation datasets are analyzed individually using the Taxa4Meta pipeline to generate taxonomic profile data for validating classification models. In some embodiments, CDI and IBD scores refer to the predicted scores of each sample as the class of CDI and IBD, respectively.

In some embodiments, a step in determining features suitable for classification of microbiome dysbiosis associated disease state and/or determination of appropriate treatment methods comprises statistical analysis. In some embodiments, comparisons between two groups are made using non-parametric Mann-Whitney-Wilcoxon two-tailed test or a suitable alternative thereof, and comparisons for more than two groups are made using non-parametric Kruskal-Wallis two-tailed test or a suitable alternative thereof. In some embodiments, multiple comparisons and pairwise Spearman or Pearson correlations are adjusted using the Benjamini-Hochberg (BH) false discovery rate (p<0.05, regarded as statistically significant), or a suitable alternative thereof.

In some embodiments, calculation of a meta-feature value is performed by: (i) determining the feature value of at least two, preferably more features, (ii) “normalizing” the feature value of each individual feature by dividing the value with a coefficient which is approximately the median value of the respective feature in a representative cohort, and (iii) calculating the median of the group of normalized gene expression values. In some embodiments, meta-feature analysis is performed as described herein.

As disclosed herein, in some embodiments, a feature shall be understood to be specifically increased in presence if the abundance level of the feature is at least about 2-fold, 4-fold, 6-fold, 8-fold, 10-fold, 15-fold, 20-fold, 30-fold, 40-fold, 50-fold, 60-fold, 70-fold, 80-fold, 90-fold, 100-fold, 1000-fold, or 10000-fold higher (or any range derivable therein) than in a reference, or in a mixture of references. References include but are not limited to, biological samples from one or more otherwise healthy individuals, biological samples from one or more individuals diagnosed with a different disease, and/or non-diarrheal biological samples from one or more individuals. In some embodiments, references can include normalized values across a cohort.

In certain algorithms a suitable threshold level is first determined for a feature. The suitable threshold level can be determined from measurements of feature presence, absence, and/or levels (e.g., quantity, activity, etc.) in one or more individuals from a test cohort. In some embodiments, median feature values in a multiple expression measurement is taken as a suitable threshold value. In some embodiments, mean feature values in a multiple expression measurement is taken as a suitable threshold value. In some embodiments, mode feature values in a multiple expression measurement is taken as a suitable threshold value. Comparison of multiple features with a threshold level can be performed as follows: 1) The individual features are compared to their respective threshold levels, 2) The number of features, the level of which is above and/or below their respective threshold level, is determined, 3) If a feature value is above its respective threshold level, then the feature level of is taken to be “above the threshold level”, 4) If a feature value is below its respective threshold level, then the feature level is taken to be “below the threshold level”.

In some embodiments, a disease classification may be determined from analysis of a sufficiently large number of features. In this context, a sufficiently large number of features means preferably 10%, 15%, 20%, 25%, 30%, 40%, 50%, 60%, 70%, 75%, 80%, 85%, 90%, 95%, 98%, 99%, or 100% of the features described by one or more binary tests.

In certain aspects, the determination of feature presence, absence, and/or levels is on a substrate that allows evaluation of RNA molecule levels from a given sample, such as a gene chip, for example but not limited to Affymetrix™ gene chip, NanoString nCounter™, Illlumina BeadChip™, etc.

In some embodiments, the determination of feature presence, absence, and/or levels is by 16S rRNA sequencing.

In some embodiments, the determination of feature presence, absence, and/or levels is by RNA sequencing.

In some embodiments, the determination of feature presence, absence, and/or levels is by whole genome sequencing, for example but not limited to, whole genome shotgun sequencing.

In other embodiments, the determination of feature presence, absence, and/or levels is done by polymerase chain reaction (PCR), for example but not limited to, real-time PCR, quantitative real time PCR, reverse transcriptase PCR, multiplexed PCR, nested PCR, long-range PCR, single-cell PCR, fast-cycling PCR, methylation-specific PCR, hot start PCR, high-fidelity PCR, in situ PCR, etc.

In some embodiments, the determination of feature presence, absence, and/or levels is performed by measuring proteins, polypeptides, metabolites, small molecules, etc. instead of nucleic based analyses (e.g., RNA and/or DNA based analyses). In some embodiments, techniques suitable for measuring the same include but are not limited to methods such as western blotting, IP-MS/MS, LC-MS/MS, NMR, PQN, ELISAs, HPLC, etc.

### B. Nucleic Acid Based Assays

Screening methods based on differential levels of genes and/or gene products are common place in the art. In accordance with one aspect of the present invention, the differential patterns of features can be determined by measuring the levels of RNA transcripts indicative of these features, or genes whose expression is modulated by the presence or absence of one or more of these features, present in a patient's biological sample (e.g., a fecal sample, swab, irrigation, mucosal biopsy, etc.). Suitable methods for this purpose include, but are not limited to, DNA sequencing, RNA sequencing, RT-PCR, Northern Blot, in situ hybridization, Southern Blot, slot-blotting, nuclease protection assay, and oligonucleotide arrays.

In some embodiments, feature absence, presence, and/or levels are determined from a biological sample obtained from a fecal sample, intestinal biopsy, intestinal swab, mucosal biopsy, and/or intestinal irrigation. In certain embodiments, feature absence, presence, and/or levels are preferably determined from a biological sample obtained from a fecal sample. In some embodiments, a biological sample may be a fixated samples (e.g., those fixed using formalin, paraformaldehyde, paraffin, etc.), blood, tears, semen, saliva, urine, feces, tissue, breast milk, lymph fluid, stool, sputum, cerebrospinal fluid, and/or supernatant from cell lysate.

In certain aspects, RNA isolated from a biological sample can be amplified to cDNA or cRNA before detection and/or quantitation. In some embodiments, isolated RNA can be either total RNA or mRNA. In some embodiments, RNA amplification can be specific or nonspecific. In some embodiments, suitable amplification methods include, but are not limited to, reverse transcriptase PCR, isothermal amplification, ligase chain reaction, and Qbeta replicase. In some embodiments, amplified nucleic acid products can be detected and/or quantitated through hybridization to labeled probes. In some embodiments, detection may involve fluorescence resonance energy transfer (FRET) or some other kind of quantum dots.

In some embodiments, amplification primers or hybridization probes for detection of presence, absence, and/or levels of a feature can be prepared from a gene sequence or obtained through commercial sources, such as Affymetrix, NanoString, Illumina BeadChip, etc. In certain embodiments a gene sequence is identical or complementary to at least 8, 10, 12, 14, 16, 18, or 20 contiguous nucleotides of a coding sequence.

In some embodiments, sequences suitable for making probes/primers for detection of a corresponding feature includes those that are identical or complementary to all or part of one or more genes specific to taxonomic units described herein. In some embodiments, sequences suitable for making probes/primers for detection of a corresponding feature includes those that are unique to one or more genes specific to taxonomic units described herein.

In some embodiments, use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In some embodiments, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene or portion of a genome of a taxonomic unit described herein. Preferably, each probe/primer has relatively high sequence complexity and does not have any ambiguous residue (undetermined “n” residues). In some embodiments, probes/primers can hybridize to a target gene, including its RNA transcripts, under stringent or highly stringent conditions. In some embodiments, because each of the features has more gene sequences, it is contemplated that probes and primers may be designed for use with any one or more of these gene sequences. For example, inosine is a nucleotide frequently used in probes or primers to hybridize to more than one sequence. It is contemplated that probes or primers may have inosine or other design implementations that accommodate recognition of more than one sequence for a particular feature.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific transcripts. It is generally appreciated that conditions can be rendered more stringent by the addition of increasing amounts of formamide.

In some embodiments, probes/primers for a gene are selected from regions which significantly diverge from the sequences of other genes. Such regions can be determined by checking the probe/primer sequences against relevant genome sequence databases. One algorithm suitable for this purpose is the BLAST algorithm. This algorithm involves first identifying high scoring sequence pairs (HSPs) by identifying short words of length (W) in the query sequence, which either match or satisfy some positive-valued threshold score (T) when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In some embodiments, quantitative RT-PCR (such as TaqMan, ABI) is used for detecting and comparing the levels of RNA transcripts in biological samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific transcripts from which the target sequence was derived may be determined for the respective cells. This direct proportionality between the concentration of the PCR products and the relative transcript abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products preferably are carried out when the PCR reactions are in the linear portion of their curves. In addition, relative concentrations of the amplifiable cDNAs preferably are normalized to some independent standard, which may be based on either internally existing RNA species or externally introduced RNA species. The abundance of a particular transcript or DNA species may also be determined relative to the average abundance of all transcript or DNA species in the sample.

In some embodiments, PCR amplification utilizes one or more internal PCR standards. The internal standard may be an abundant housekeeping gene in a cell. These standards may be used to normalize expression and/or abundance levels so that the expression and/or abundance levels of different features can be compared directly. A person of ordinary skill in the art would know how to use an internal standard to normalize expression and/or abundance levels.

A problem inherent in clinical samples is that they are generally of variable quantity and/or quality. In some embodiments, this problem can be overcome if the RT-PCR is performed as a relative quantitative RT-PCR with an internal standard in which the internal standard is an amplifiable nucleic acid fragment that is similar or larger than the target nucleic acid fragment and in which the abundance of the nucleic acid fragment encoding the internal standard is roughly 5-100 fold higher than the nucleic acid fragment encoding the target. This assay measures relative abundance, not absolute abundance of the respective nucleic acid species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target nucleic acid fragment.

Nucleic acid arrays can also be used to detect and compare the differential presence, absence, or levels of microbiome dysbiosis features. Probes suitable for detecting the corresponding features can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same targets representative of one or more features. Multiple probes for the same feature can be used on a single nucleic acid array. Probes for other features can also be included in the nucleic acid array. Probe combinations suitable for delineation of healthy, CDI, IBS, IBD UC, and/or IBD CD can be included on a nucleic acid array. The probe density on the array can be in any range. In some embodiments, the density may be 50, 100, 200, 300, 400, 500 or more probes/cm2.

Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the presence, absence, and/or levels of one or more features with respect to diagnostic, prognostic, and treatment methods of the disclosure.

The present disclosure may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality genes and/or gene products and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect absence, presence, or levels of one or more features; consequently, methods and compositions may be used with a variety of different types of genes and/or gene products.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO 0138580; WO 0168255; WO 03020898; WO 03040410; WO 03053586; WO 03087297; WO 03091426; WO 03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448, all of which are specifically incorporated by reference.

In some embodiments, nuclease protection assays are used to quantify RNAs derived from a biological sample. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

In some embodiments, the presence, absence, and/or levels of one or more features are determined from a biological sample using 3′ RNA sequencing, using products such as Lexogen QuantSeq™, QioSeq UPX 3′ Transcriptome, etc. In some embodiments, 3′ RNA sequencing does not require transcripts to be fragmented before reverse transcription, and cDNAs are reverse transcribed only from the 3′ RNA sequencing end of the transcripts, resulting in only one copy of cDNA for each transcript, resulting in a direct 1:1 ratio between RNA and cDNA copy numbers.

In some embodiments, gene expression is determined from a biological sample using specific targeted sequencing, using products such as BioSpyder Temp0-Seq®, Ion Ampliseq™ Transcriptome, etc. In some embodiments, specific targeted sequencing targets RNA sequences by hybridization to DNA oligos followed by removal of unhybridized oligos and amplification of remaining products.

C. Proteins, Polypeptide, Metabolites, Etc. Based Assays

In other embodiments, the differential features (e.g., taxonomic and/or metabolic pathway biomarkers) can be determined by measuring levels of polypeptides encoded by components of the microbiome in a biological sample (e.g., a fecal sample, intestinal swap, intestinal biopsy, intestinal irrigation sample, etc.). Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded or implicated by one or more features described herein.

One example of a method suitable for detecting the levels of target proteins in biological samples is ELISA. In an exemplifying ELISA, antibodies capable of binding to the target proteins encoded by the genome of one or more features are immobilized onto a selected surface exhibiting protein affinity, such as wells in a polystyrene or polyvinylchloride microtiter plate. Then, samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Proper extraction procedures can be used to separate the target proteins from potentially interfering substances.

In another ELISA embodiment, one or more samples containing the target proteins reflective of one or more features are immobilized onto the well surface and then contacted with antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Non-limiting examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In some embodiments, a label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with a labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

In some embodiments, another suitable method is RIA (radioimmunoassay). An example of RIA is based on the competition between radiolabeled-polypeptides and unlabeled polypeptides for binding to a limited quantity of antibodies. Suitable radiolabels include, but are not limited to, 1125. In some embodiments, a fixed concentration of I125-labeled polypeptide is incubated with a series of dilution of an antibody specific to the polypeptide. When the unlabeled polypeptide is added to the system, the amount of the I125-polypeptide that binds to the antibody is decreased. A standard curve can therefore be constructed to represent the amount of antibody-bound I125-polypeptide as a function of the concentration of the unlabeled polypeptide. From this standard curve, the concentration of the polypeptide in unknown samples can be determined. Various protocols for conducting RIA to measure the levels of polypeptides in a sample are well known in the art.

In some embodiments, suitable antibodies for biomarker detection include, but are not limited to, polyclonal antibodies, monoclonal antibodies, chimeric antibodies, humanized antibodies, single chain antibodies, Fab fragments, and fragments produced by a Fab expression library.

In some embodiments, antibodies can be labeled with one or more detectable moieties to allow for detection of antibody-antigen complexes. In some embodiments, detectable moieties can include compositions detectable by spectroscopic, enzymatic, photochemical, biochemical, bioelectronic, immunochemical, electrical, optical or chemical means. In some embodiments, detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a sample comprising a representative population of a microbiome.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In some embodiments, the levels of polypeptides in a biological sample can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

In some embodiments, the levels of polypeptides and/or metabolites in a biological sample can be determined by IP-MS/MS and/or HPLC.

### D. Methods of Delineation Between Disease Classification States.

In certain embodiments, one or more features identified herein can be used to delineate between disease classification states, and/or to provide stake holders with a basis for prescribing one or more appropriate methods of treatment.

In certain embodiments, one or more features are the presence, absence, and/or level of one or more a metabolic pathways.

In certain embodiments, one or more features are the presence, absence, and/or level of one or more taxonomic unit.

In certain embodiments, one or more features are the presence, absence, and/or level of one or more taxonomic units represented by one or more particular bacteria.

In certain embodiments, one or more features associated with the presence, absence, and/or level of one or more a metabolic pathways is used in conjunction with one or more features associated with the presence, absence, and/or level of one or more taxonomic units. In certain embodiments, one or more features associated with a feature are described in any one of tables 1-19.

In certain embodiments, a disease classification can be determined by the presence, absence, or relative level of at least one of AST-PWY (L-arginine degradation II (AST pathway)), ECASYN-PWY (enterobacterial common antigen biosynthesis), THREOCAT-PWY (superpathway of L-threonine metabolism), PPGPPMET-PWY (ppGpp biosynthesis), PWYO-1338 (polymyxin resistance), PWY-6263 (superpathway of menaquinol-8 biosynthesis II), PWY-7371 (1,4-dihydroxy-6-naphthoate biosynthesis II), PWY-7374 (1,4-dihydroxy-6-naphthoate biosynthesis I), P221-PWY (octane oxidation), PWY-6749 (CMP-legionaminate biosynthesis I), PWY-7456 (mannan degradation), NONMEVIPP-PWY (methylerythritol phosphate pathway I), PWY-5097 (L-lysine biosynthesis VI), PWY-5505 (L-glutamate and L-glutamine biosynthesis), PWY-6122 (5-aminoimidazole ribonucleotide biosynthesis II), PWY-7663 (gondoate biosynthesis (anaerobic)), THRESYN-PWY (superpathway of L-threonine biosynthesis), HEMESYN2-PWY (heme biosynthesis II (anaerobic)), PWY-5304 (superpathway of sulfur oxidation (archaea), PWY-6478 (GDP-D-glycero-alpha-D-manno-heptose biosynthesis), PWY-7198 (pyrimidine deoxyribonucleotides de novo biosynthesis IV), and/or PWY-7210 (pyrimidine deoxyribonucleotides biosynthesis from CTP).

In certain embodiments, an increased abundance relative to an appropriate control of at least one of or all of AST-PWY (L-arginine degradation II (AST pathway)), ECASYN-PWY (enterobacterial common antigen biosynthesis), THREOCAT-PWY (superpathway of L-threonine metabolism), PPGPPMET-PWY (ppGpp biosynthesis), and/or PWYO-1338 (polymyxin resistance) is associated with CDI causative diarrhea. In certain embodiments, following detection of one or more of the indicative features, an individual is then treated accordingly.

In certain embodiments, an increased abundance relative to an appropriate control of at least one of or all of PWY-6263 (superpathway of menaquinol-8 biosynthesis II), PWY-7371 (1,4-dihydroxy-6-naphthoate biosynthesis II), PWY-7374 (1,4-dihydroxy-6-naphthoate biosynthesis I), P221-PWY (octane oxidation), PWY-6749 (CMP-legionaminate biosynthesis I), and/or PWY-7456 (mannan degradation) is associated with IBD UC causative diarrhea. In certain embodiments, following detection of one or more of the indicative features, an individual is then treated accordingly.

In certain embodiments, an increased abundance relative to an appropriate control of at least one of or all of NONMEVIPP-PWY (methylerythritol phosphate pathway I), PWY-5097 (L-lysine biosynthesis VI), PWY-5505 (L-glutamate and L-glutamine biosynthesis), PWY-6122 (5-aminoimidazole ribonucleotide biosynthesis II), PWY-7663 (gondoate biosynthesis (anaerobic)), THRESYN-PWY (superpathway of L-threonine biosynthesis), HEMESYN2-PWY (heme biosynthesis II (anaerobic)), PWY-5304 (superpathway of sulfur oxidation (archaea), PWY-6478 (GDP-D-glycero-alpha-D-manno-heptose biosynthesis), PWY-7198 (pyrimidine deoxyribonucleotides de novo biosynthesis IV), and/or PWY-7210 (pyrimidine deoxyribonucleotides biosynthesis from CTP) is associated with IBD causative. In certain embodiments, following detection of one or more of the indicative features, an individual is then treated accordingly.

In certain embodiments, methods described herein for classification of a disease state may comprise, or expressly does not comprise, detection and/or quantification of Adlercreutzia, or a metabolic pathway associated therewith.

In certain embodiments, methods described herein for classification of a disease state may comprise, or expressly does not comprise, detection and/or quantification of Agathobaculum, or a metabolic pathway associated therewith.

In certain embodiments, methods described herein for classification of a disease state may comprise, or expressly does not comprise, detection and/or quantification of Akkermansia, or a metabolic pathway associated therewith.

In certain embodiments, methods described herein for classification of a disease state may comprise, or expressly does not comprise, detection and/or quantification of Alistipes, or a metabolic pathway associated therewith.

In certain embodiments, methods described herein for classification of a disease state may comprise, or expressly does not comprise, detection and/or quantification of Anaerostipes, or a metabolic pathway associated therewith.

In certain embodiments, methods described herein for classification of a disease state may comprise, or expressly does not comprise, detection and/or quantification of Bacteroides, or a metabolic pathway associated therewith.

In certain embodiments, methods described herein for classification of a disease state may comprise, or expressly does not comprise, detection and/or quantification of Barnesiella, or a metabolic pathway associated therewith.

In certain embodiments, methods described herein for classification of a disease state may comprise, or expressly does not comprise, detection and/or quantification of Bifidobacterium, or a metabolic pathway associated therewith.

In certain embodiments, methods described herein for classification of a disease state may comprise, or expressly does not comprise, detection and/or quantification of Bilophila, or a metabolic pathway associated therewith.

In certain embodiments, methods described herein for classification of a disease state may comprise, or expressly does not comprise, detection and/or quantification of Blautia, or a metabolic pathway associated therewith.

In certain embodiments, methods described herein for classification of a disease state may comprise, or expressly does not comprise, detection and/or quantification of Butyricimonas, or a metabolic pathway associated therewith.

In certain embodiments, methods described herein for classification of a disease state may comprise, or expressly does not comprise, detection and/or quantification of Clostridium, or a metabolic pathway associated therewith.

In certain embodiments, methods described herein for classification of a disease state may comprise, or expressly does not comprise, detection and/or quantification of Clostridium IV, or a metabolic pathway associated therewith.

In certain embodiments, methods described herein for classification of a disease state may comprise, or expressly does not comprise, detection and/or quantification of Clostridium XIVa, or a metabolic pathway associated therewith.

In certain embodiments, methods described herein for classification of a disease state may comprise, or expressly does not comprise, detection and/or quantification of Clostridium XVIII, or a metabolic pathway associated therewith.

In certain embodiments, methods described herein for classification of a disease state may comprise, or expressly does not comprise, detection and/or quantification of Clostridium sensu stricto, or a metabolic pathway associated therewith.

In certain embodiments, methods described herein for classification of a disease state may comprise, or expressly does not comprise, detection and/or quantification of Collinsella, or a metabolic pathway associated therewith.

In certain embodiments, methods described herein for classification of a disease state may comprise, or expressly does not comprise, detection and/or quantification of Coprococcus, or a metabolic pathway associated therewith.

In certain embodiments, methods described herein for classification of a disease state may comprise, or expressly does not comprise, detection and/or quantification of Dialister, or a metabolic pathway associated therewith.

In certain embodiments, methods described herein for classification of a disease state may comprise, or expressly does not comprise, detection and/or quantification of Dorea, or a metabolic pathway associated therewith.

In certain embodiments, methods described herein for classification of a disease state may comprise, or expressly does not comprise, detection and/or quantification of Enterococcus, or a metabolic pathway associated therewith.

In certain embodiments, methods described herein for classification of a disease state may comprise, or expressly does not comprise, detection and/or quantification of Erysipelatoclostridium, or a metabolic pathway associated therewith.

In certain embodiments, methods described herein for classification of a disease state may comprise, or expressly does not comprise, detection and/or quantification of Eubacterium, or a metabolic pathway associated therewith.

In certain embodiments, methods described herein for classification of a disease state may comprise, or expressly does not comprise, detection and/or quantification of Faecalibacterium, or a metabolic pathway associated therewith.

In certain embodiments, methods described herein for classification of a disease state may comprise, or expressly does not comprise, detection and/or quantification of Flavonifractor, or a metabolic pathway associated therewith.

In certain embodiments, methods described herein for classification of a disease state may comprise, or expressly does not comprise, detection and/or quantification of Fusicatenibacter, or a metabolic pathway associated therewith.

In certain embodiments, methods described herein for classification of a disease state may comprise, or expressly does not comprise, detection and/or quantification of Fusobacterium, or a metabolic pathway associated therewith.

In certain embodiments, methods described herein for classification of a disease state may comprise, or expressly does not comprise, detection and/or quantification of Gemmiger, or a metabolic pathway associated therewith.

In certain embodiments, methods described herein for classification of a disease state may comprise, or expressly does not comprise, detection and/or quantification of Haemophilus, or a metabolic pathway associated therewith.

In certain embodiments, methods described herein for classification of a disease state may comprise, or expressly does not comprise, detection and/or quantification of Intestinibacter, or a metabolic pathway associated therewith.

In certain embodiments, methods described herein for classification of a disease state may comprise, or expressly does not comprise, detection and/or quantification of Lachnoclostridium, or a metabolic pathway associated therewith.

In certain embodiments, methods described herein for classification of a disease state may comprise, or expressly does not comprise, detection and/or quantification of Lachnospiraceae, or a metabolic pathway associated therewith.

In certain embodiments, methods described herein for classification of a disease state may comprise, or expressly does not comprise, detection and/or quantification of Lachnospiraceae incertae sedis, or a metabolic pathway associated therewith.

In certain embodiments, methods described herein for classification of a disease state may comprise, or expressly does not comprise, detection and/or quantification of Lactobacillus, or a metabolic pathway associated therewith.

In certain embodiments, methods described herein for classification of a disease state may comprise, or expressly does not comprise, detection and/or quantification of Odoribacter, or a metabolic pathway associated therewith.

In certain embodiments, methods described herein for classification of a disease state may comprise, or expressly does not comprise, detection and/or quantification of Parabacteroides, or a metabolic pathway associated therewith.

In certain embodiments, methods described herein for classification of a disease state may comprise, or expressly does not comprise, detection and/or quantification of Paraprevotella, or a metabolic pathway associated therewith.

In certain embodiments, methods described herein for classification of a disease state may comprise, or expressly does not comprise, detection and/or quantification of Parasutterella, or a metabolic pathway associated therewith.

In certain embodiments, methods described herein for classification of a disease state may comprise, or expressly does not comprise, detection and/or quantification of Phascolarctobacterium, or a metabolic pathway associated therewith.

In certain embodiments, methods described herein for classification of a disease state may comprise, or expressly does not comprise, detection and/or quantification of Prevotella, or a metabolic pathway associated therewith.

In certain embodiments, methods described herein for classification of a disease state may comprise, or expressly does not comprise, detection and/or quantification of Romboutsia, or a metabolic pathway associated therewith.

In certain embodiments, methods described herein for classification of a disease state may comprise, or expressly does not comprise, detection and/or quantification of Roseburia, or a metabolic pathway associated therewith.

In certain embodiments, methods described herein for classification of a disease state may comprise, or expressly does not comprise, detection and/or quantification of Ruminococcus, or a metabolic pathway associated therewith.

In certain embodiments, methods described herein for classification of a disease state may comprise, or expressly does not comprise, detection and/or quantification of Ruminococcus2, or a metabolic pathway associated therewith.

In certain embodiments, methods described herein for classification of a disease state may comprise, or expressly does not comprise, detection and/or quantification of Ruthenibacterium, or a metabolic pathway associated therewith.

In certain embodiments, methods described herein for classification of a disease state may comprise, or expressly does not comprise, detection and/or quantification of Ruminococcus, or a metabolic pathway associated therewith.

In certain embodiments, methods described herein for classification of a disease state may comprise, or expressly does not comprise, detection and/or quantification of Schaalia, or a metabolic pathway associated therewith.

In certain embodiments, methods described herein for classification of a disease state may comprise, or expressly does not comprise, detection and/or quantification of Streptococcus, or a metabolic pathway associated therewith.

In certain embodiments, methods described herein for classification of a disease state may comprise, or expressly does not comprise, detection and/or quantification of Sutterella, or a metabolic pathway associated therewith.

In certain embodiments, methods described herein for classification of a disease state may comprise, or expressly does not comprise, detection and/or quantification of Turicibacter, or a metabolic pathway associated therewith.

In certain embodiments, methods described herein for classification of a disease state may comprise, or expressly does not comprise, detection and/or quantification of Veillonella, or a metabolic pathway associated therewith.

In certain aspects, methods disclosed herein can relate to a system for performing such methods, the system comprising (a) apparatus or device for storing data regarding feature levels of one or more microbiome components; (b) apparatus or device for determining feature levels of at least one feature; (c) apparatus or device for comparing feature levels of a first feature with a predetermined first threshold value and/or test value; (d) apparatus or device for determining feature level of at least one second or more features; and (e) computing apparatus or device programmed to provide treatment with an appropriate methodology if the data indicates altered feature levels or activity of said first feature as compared to the predetermined first threshold value and/or test value, and, alternatively or in concert, expression level and/or activity of said second or more features as compared to the predetermined second or more feature threshold level and/or test value.

A person skilled in the art readily appreciates that an accurate prognosis can be given or determined if a sufficiently large number of feature levels are analyzed and compared to an appropriate control. In some embodiments, accurate prognosis can facilitate determination of disease recurrence and/or appropriate therapies to provide, including a particular therapy of any kind, such as an antibiotic therapy.

In some embodiments, feature levels and/or patterns can also be compared by using one or more ratios between feature abundance levels associated with an otherwise healthy microbiome and/or one or more dysbiosed microbiomes. Other suitable measures or indicators can also be employed for assessing the relationship or difference between different feature patterns.

In some embodiments, one or more of the features can be used to determine whether a patient with a diarrheal disorder should be treated with antimicrobials and/or antibiotics. In certain embodiments, a pattern of features in a patient fecal sample and/or other microbiome samples may be used to evaluate a patient to determine whether they are likely to respond to one or more therapeutic interventions. In some embodiments, likeliness of a therapeutic response for the patient may be considered with respect to an individual that lacks the particular feature pattern of the patient.

In some embodiments, a subject's (e.g., a patient's) feature levels can be compared to reference feature levels using various methods. In some embodiments, reference levels can be determined using expression levels of a reference based on otherwise healthy patients, all types of FGID patients, and/or all types of CDI, IBS, and/or IBD patients. In some embodiments, reference levels can be based on an internal reference such as a gene, metabolic pathway, and/or microbe that is present ubiquitously. In some embodiments, comparison can be performed using the fold change or the absolute difference between the feature levels to be compared. In some embodiments, one or more taxonomic and/or metabolic features can be used in the comparison.

In some embodiments, it is contemplated that 1, 2, 3, 4, 5, 6, 7, 8, 9, 10, 11, 12, 13, 14, 15, 16, 17, 18, 19, 20, 21, 22, 23, 24, and/or 25 features may be compared to each other and/or to a reference that is internal or external. In some embodiments, it is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100 features may be compared to each other and/or to a reference that is internal or external. In some embodiments, it is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, and/or 200 features may be compared to each other and/or to a reference that is internal or external.

In certain embodiments, comparisons or results from comparisons may reveal or be expressed as x-fold increase or decrease in expression relative to a standard or relative to another feature or relative to the same feature but in a different patient cohort (e.g., a disease patient and/or cohort compared to an appropriate health control). In some embodiments, patients with a particular disease diagnosis may have a relatively high level of feature presentation (e.g., over representation) or relatively low level of feature presentation (e.g., under representation) when compared to patients with a different disease diagnosis and/or otherwise healthy patients, or vice versa.

Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000, or greater than 1000% difference, or any range derivable therein. In some embodiments, a fold level change for one or more features may not be calculatable, as one or more features may be absent in one or more disease and/or control patients and/or cohorts (e.g., dividing by zero).

Other ways to express relative expression levels are by normalized or relative numbers such as 0, 0.00001, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein.

In certain embodiments, a feature may be ranked in importance and/or otherwise identified according to a random forest feature rank mean decrease in accuracy. For example, a feature may be considered more integral for appropriate disease classification as a function of the random forest feature rank mean decrease in accuracy. In some embodiments, a higher random forest feature mean decrease in value means the feature has a greater potential disease classification value when compared to a feature with a lower value. In certain embodiments, a feature random forest feature rank mean decrease in accuracy may be 0.00001, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, or any range derivable therein.

In some embodiments, algorithms, such as the weighted voting programs, can be used to facilitate the evaluation of feature levels. In addition, in some embodiments, other clinical evidence can be combined with a feature-based test to reduce the risk of false evaluations. In some embodiments, other molecular based evaluations may be considered. In some embodiments, patient questionnaires may be considered. In some embodiments, patient medical histories may be considered. In some embodiments, patient endoscopy results may be considered.

In some embodiments, any biological sample from a patient that accurately represents the microbiome may be used to evaluate the presence, absence, and/or level of any feature discussed herein. In some embodiments, a biological sample from a fecal sample is used. In some embodiments, a biological sample from an endoscopy is used. In some embodiments, a biological sample from a mucosal biopsy is used. In some embodiments, a biological sample from intestinal fluid is used. Evaluation of a biological sample may involve, though it need not involve, panning (enriching) for microbiome components or isolation of specific microbes.

### III. Methods of Therapeutic Intervention

In some embodiments, methods described herein are not limited to intestinal disorders, but are applicable to other microbiome dysbiosis associated disorders.

In certain embodiments, methods of treatment of intestinal disorders are based on features (e.g., taxa and/or metabolic pathways) identified by Taxa4Meta mediated diverse 16S data analysis.

In certain aspects, provided herein are methods for treating a subject determined to have CDI, IBS, IBD UC, and/or IBD CD based on a predetermined profile of one or more taxonomic and/or metabolic pathway features disclosed herein.

In certain embodiments, provided herein are methods for identifying features associated with diseases associated with microbiome dysbiosis, for example but not limited to CDI, IBS, IBD UC, IBD CD, antibiotic-associated diarrhea (AAD), celiac disease, food allergies, autoimmune disease, cancer, and/or graft versus host disease.

In some embodiments, an appropriate therapeutic agent is a small molecule, a biologic (e.g., an antibody, a recombinant protein, a cell therapy, etc.), a microbiota therapy (e.g., fecal transplant, fecal microbiota therapy, etc.), a mineral, a vitamin, a dietary restriction, a life style restriction and/or behavioral therapy.

In some embodiments, an appropriate therapeutic intervention for treating a subject that has received a CDI disease classification include but are not limited to administration of: vancomycin, fidaxomicin, bezlotoxumab, metronidazole (less preferred), fecal microbiota therapy (FMT) (e.g., particularly in cases of recurrent CDI), and/or microbiota consortia products.

In some embodiments, an appropriate therapeutic intervention for treating a subject that has received an IBD disease classification include but are not limited to administration of: anti-inflammatory drugs (e.g., for reduction of digestive tract inflammation), sulfasalazine, corticosteroids, immune suppressants (e.g., to prevent the autoimmune attacks), azathioprine, antibiotics (e.g., to ameliorate bacterial infections), ciprofloxacin, metronidazole, TNF signaling pathway antagonists, Cimzia, α4β7 integrin antagonists, Entyvio (vedolizumab), Humira (adalimumab), Remicade (infliximab), Simponi (golimumab), Stelara (ustekinumab), anti-diarrheal agents (e.g., to prevent diarrhea and ameliorate associated symptoms), loperamide, diphenoxylate, cholestyramine, analgesics (e.g., to reduce pain and ameliorate associated symptoms), acetaminophen, vitamin and/or mineral supplements, vitamin D, and/or calcium.

In some embodiments, an appropriate therapeutic intervention for treating a subject that has received an IBS disease classification include but are not limited to administration of: bezlotoxumab, anti-diarrheal agents (e.g., to prevent diarrhea and/or ameliorate associated symptoms), loperamide, cholestyramine, colestipol, anticholinergics (e.g., to relieve spasms), dicyclomine, tricyclic antidepressants (e.g., to relieve depression and severe pain), imipramine, desipramine, selective serotonin reuptake inhibitors (SSRIs) (e.g., to relieve depression, pain and/or constipation), fluoxetine, paroxetine, anticonvulsants (e.g., to relieve pain and/or bloating), pregabalin, and/or gabapentin.

Therapy provided herein may comprise administration of a combination of therapeutic agents, such as for example, a first therapy (e.g., antimicrobials) and a second therapy (e.g., dietary restrictions). The therapies may be administered in any suitable manner known in the art. For example, the first and second treatment may be administered sequentially (at different times) or concurrently (at the same time).

In some aspects, the first therapy and the second therapy are administered substantially simultaneously. In some aspects, the first therapy and the second therapy are administered sequentially. In some aspects, the first therapy, the second therapy, and a third therapy are administered sequentially. In some aspects, the first therapy is administered before administering the second therapy. In some aspects, the first therapy is administered after administering the second therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

Therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose (also “effective amount” or “therapeutically effective amount”) is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

Various combinations of antimicrobial and/or antibiotic agents or compounds may be employed, for example an antimicrobial is “A” and an additional therapeutic agent is “B” (or a combination of such agents and/or compounds), and given as part of a therapeutic regimen, for example:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a therapeutic compounds or agents to a patient will follow general protocols for the administration of such compounds, taking into account the toxicity, if any, of a therapy. It is expected that treatment cycles would be repeated as necessary. It also is contemplated that various standard therapies, as well as surgical intervention, may be applied in combination with a described therapy.

### IV. Kits

In some embodiments, the present invention also concern kits containing compositions of the disclosure or compositions to implement methods of the disclosure. In some embodiments, a kit comprises one or more bacteria from Cluster 2, 4, and/or 5 enterotype.

In one embodiment, there is a kit, comprising (a) one or more species selected from the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037; (b) one or more species selected from the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified 074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV; and/or (c) one or more species selected from the following listed bacterial taxa: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available_2, Bilophila, Barnesiella, Dialister, Butyricimonas, Clostridium, Intestinibacter, Paraprevotella, Haemophilus, Clostridium IV, Phascolarctobacterium, and Flavonifractor.

In some aspects, kits can be used to evaluate the presence of one or more bacteria and/or bacterial taxa. In some aspects, kits can be used to detect, for example, absence, presence, and/or level of one or more features described herein. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 100, 132, 500, 1,000 or more probes, primers or primer sets, synthetic molecules or inhibitors, or any value or range and combination derivable therein.

In some embodiments, a kit can be prepared from readily available materials and reagents. For example, such kits can comprise any one or more of the following materials: enzymes, reaction tubes, buffers, detergent, primers, probes, antibodies. In some embodiments, a kit allows a practitioner to obtain biological samples. In another preferred embodiment these kits include the needed apparatus for performing RNA extraction, RT-PCR, oligonucleotide quantification, protein and/or metabolite quantification, and/or gel electrophoresis. Instructions for performing associated assays can also be included in a kit.

In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24 and/or 25 features. In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100 features. In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, and/or 200 or more features.

In some embodiments, a kit is housed in a container. Kits may further comprise instructions for using the kit for assessing expression, means for converting the expression data into expression values and/or means for analyzing expression values to generate prognosis. Agents in a kit for measuring biomarker expression may comprise a plurality of PCR probes and/or primers for qRT-PCR and/or a plurality of antibody or fragments thereof for assessing expression of biomarkers. In another embodiment, agents in a kit for measuring biomarker expression may comprise an array of polynucleotides complementary to mRNAs of biomarkers identified herein. Possible means for converting expression data into expression values and for analyzing expression values to generate scores that predict survival or prognosis may be also included.

In some embodiments, a kit may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for copy number or expression of one or more biomarkers.

Any aspect of the disclosure involving specific taxanomic profile and/or metabolic pathway biomarker by name is contemplated also to cover aspects involving biomarkers whose characteristics are at least 80, 81, 82, 83, 84, 85, 86, 87, 88, 89, 90, 91, 92, 93, 94, 95, 96, 97, 98, or 99% identical to the specified taxanomic profile and/or metabolic pathway.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods of Classifying and Treating Inflammatory Bowel Disease

The present example concerns methods and compositions for classifying and treating IBD. The excessive immune response in the GI tract is addressed herein by categorizing an individual at risk for IBD or suspected of having IBD based on a tax profile of their microbiome.

IBD microbiome clusters: The data show that new IBD-clusters represent a potential disease contributor to UC and CD in adults and children. This is based on the consideration that IBD is a notable GI disease where clinical microbiome surveys have provided promising insights into microbiome-associations and mechanisms. However, systematic review of these largely single-site cohort studies have demonstrated inconsistent findings, in large part due to variations in methods for data generation and analysis as they introduce significant bias for cross-comparisons. Because the utility of pan-microbiome profiling has not yet been tested for disease classification, it was explored whether this approach improved classifier scores by merging core microbiome communities corrected for demographic and technical bias. In a comprehensive re-analysis of 4,491 IBD and matched healthy and IBS disease control subjects from 36 clinical cohorts spanning North America, Europe and Australasia, the inventors used several different bioinformatic and statistical methods to identity IBD-dominant microbiome signatures. Using hierarchical clustering analysis for family abundance profiles it was demonstrated that UC (n=1,095) and CD (n=874) patients generally clustered with controls (n=1,678) and IBS patients (n=844); whereas these patients could be separated forming a distinct IBD cluster when analyzed by DMM clustering, forming dominant IBD clusters (Clusters 1 & 3) versus Clusters 2, 4 and 5 that are predominantly comprised of healthy and IBS subjects. These IBD enterotypes were statistically validated by Chi squared analysis that demonstrated highly significant adjusted residual scores supporting the concept that IBD dominant microbiota clusters are evident. Moreover, using a random forest supervised learning model it was demonstrated that the top IBD-specific taxa (that included several unclassified species not identified using other taxonomical profilers) separated IBD and control subjects appropriately in a PCA plot and predominately associated with IBD cluster 1 and microbiome cluster 3. By contrast, it was found that CDI patients are consistently diagnosed with a microbiome cluster 3 type, dominated by dysbiotic taxa and pathobiome. Finally, in a Chi squared analysis1 of clinical responders to FMT, significant longitudinal microbiome cluster switching and clinical remission followed treatment of IBD patients with non-IBD associated clusters 2, 4, and/or 5 donors.

Methods used to determine disease clusters. The inventors used specialized 16S-based methods (Taxa4Meta) to classify taxonomic abundance and assign an individual's enterotype, which the inventors validated using deeper metagenomic sequencing for species and strain identification, as well as deciphering microbial genes and metabolic pathways linked with disease clusters that they showed are significantly associated with IBD symptoms and treatment outcomes. As a primary outcome measure, the inventors assigned microbiome clusters (Clusters 2, 4 & 5 (healthy) vs. IBD-specific cluster 1 and CDI- and IBD-associated cluster 3) to individuals, as well as to longitudinal specimens in order to establish cluster stability with time and disease activity. Significant microbiome cluster switching was observed from IBD clusters 1 and 3, towards healthy clusters with clinical remission following successful FMT, whereas this is not evident with a disease flare up. The inventors applied principal coordinates analysis (PCA) to Bray-Curtis and abundance-weighted Jaccard distances at bacterial species-level and PERMANOVA statistical tests to evaluate the level of microbial similarity (Bray-Curtis and abundance-weighted Jaccard distances) with disease subtype and activity. Wilcoxon or Mann-Whitney nonparametric tests were used to understand whether microbiome clusters are shifted with a new onset IBD disease diagnosis or a relapse, and at each longitudinal time point.

Determination of microbiome cluster dominance: Taxa4Meta was applied to 16S rDNA sequence data and collapsed species tables were merged into our comprehensive microbiome compositional template containing 3,991 IBD and matched control subjects to determine how subtype impacts cluster dominance and how this changed with disease onset and longitudinal stability. Taxonomical profiles were validated using MetaPhlAn2 for matched WGS data available. Both reference-based and de novo approaches were used for profiling the WGS data: for reference-based analysis, MetaPhlAn2 was utilized for species identification considering its high precision and recall independent of sequencing depth, and HUMAnN2 was adopted for profiling metabolic pathways for each species; for de novo analysis, the inventors individually assembled shotgun data after quality filtering and error correction using megahit, and generated non-redundant assemblies via sequence clustering/OLC assembly/bin de-replication for further comparative metagenomics analysis using the Anvi'o platform with its recommended workflow. Specifically, sequence reads were considered with minimal mapQ of 2 termed single aligned reads for profiling the abundance of contigs, and these were used in the metagenomic binning with tools BinSanity, CONCOCT or/and human-guided refinement in Anvi'o. Similar settings were used in Orange or H2O.ai platforms for constructing the shotgun-based IBD validation of Taxa4Meta 16S risk profiles with results of taxa abundance generated in MetaPhlAn2 alone or in combination with pathway profiles from HUMAnN2 and/or gene abundance from de novo analysis. Selected features from 16S and WGS-based IBD clusters were compared; common features (concordant genus & species) in combination with pathways and genes identified were mined for specific metabolic traits linked to IBD clusters and clinical metadata in baseline and longitudinal specimens. Deep sequencing data and the StrainFinder pathon package were also used to evaluate the impact of cluster switching at the strain-level. StrainFinder aligns all of the metagenomic reads from a sample against a reference sequence, tabulates the SNPs at every position of the alignment, and generates maximum likelihood estimates of strain genotypes and their frequencies. To evaluate cluster switching at the strain-level, StrainFinder analysis of WGS data was performed to assess the overall impact on percent of strain composition that is stable in IBD clusters over time. This approach allowed determination of the impact of cluster switching and the specific taxa that shape susceptibility clusters over time, for example loss of Fecalibacteria strains and the unstable emergence of Enterbacteriales and pathobiome are significant features associated with clinical IBD symptoms:

Summary: Microbiome science is still a rapidly evolving field with advances in sequencing strategies and bioinformatics facilitating our understanding of host-microbiota interactions. Nevertheless, these technological advances should not dissuade the use of retrospective microbiome data because these projects could collectively provide critical insight into disease-specific associations. However, individual clinical microbiome surveys often utilize cohort-specific sequencing platforms, 16S primer regions and bioinformatics pipelines, which the inventors systematically demonstrate requires a consolidated bioinformatics approach to mitigate technological and demographic bias, as well as taxonomic misclassification. The findings show that previous IBD microbiome meta-analyses have not adequately addressed these limitations. The inventors utilized Taxa4Meta, a bioinformatics pipeline for accurate taxonomic profiling after systematically benchmarking sequence orientation and length so that data output can be reliably utilized from different 16S variable regions. Given the challenges of accurately merging OTU tables generated from different 16S variable regions, the inventors collapsed taxonomic annotations of Taxa4Meta feature profiles as a new binning approach to facilitate meta-analysis of diverse 16S amplicon data. Taxa4Meta was then applied to comprehensively re-analyze diverse 16S datasets generated from multiple retrospective IBD cohorts investigated across three continents. Supervised classification distinguished IBD from other diarrheal patients who are difficult to diagnose because of overlapping GI symptoms. This approach facilitated construction of a prototypic diagnostic workflow based on disease-specific pan-microbiome biomarkers and the discovery of new IBD-clusters comprising disease-associated taxa that will demonstrate to be associated with human microbiome immaturity and lack of immune tolerance.

Patient cohorts and clinical definitions. A meta-analysis was conducted using 27 patient cohorts with available raw 16S sequencing data that had been previously published. An additional 13 patient cohorts were utilized for independent validation purposes. For the majority of cohorts, sample grouping information was available after NCBI BioSample registration during data deposition. For CDI cohorts, symptoms of diarrhea, PCR-based toxin gene detection, and enzyme immunoassay tests for C. difficile toxins were commonly reported for diagnosis per the 2017 IDSA/AGA guidelines. For IBD diagnosis, colonoscopy, Montreal classification, and disease activity index were used in the majority of cohorts. Disease severity in UC was measured using several quantitative methods, including the Mayo score and the Simple Clinical Colitis Activity Index, which have been found to correlate well with endoscopic disease activity. For CD diagnosis, the Crohn's Disease Activity Index is commonly used. It should be noted, however, that the disease status was not clearly indicated for all patients and specimens. Therefore, data collected during active and remission stages were combined for meta-analysis and classification. Diagnosis of IBS and its disease status relied on a questionnaire and the Rome III criteria.

Microbiome meta-analysis of diarrheal microbiome datasets. Each diarrheal dataset was processed through the Taxa4Meta pipeline using optimal taxonomic thresholds for each 16S variable region. The relative abundance of collapsed species profiles generated from each Taxa4Meta OTU count table was used without rarefaction, but a minimum of 1,000 reads per sample was required. If a species was assigned by Taxa4Meta-BLCA, the taxonomic lineage from NCBI 16S RefSeq was adopted for that species to avoid any inconsistencies in the taxonomic lineage. The merging of Taxa4Meta collapsed profiles from all datasets was based on the taxonomic lineages.

### Embodiments of the Disclosure

The following provides demonstrations of embodiments of the disclosure.

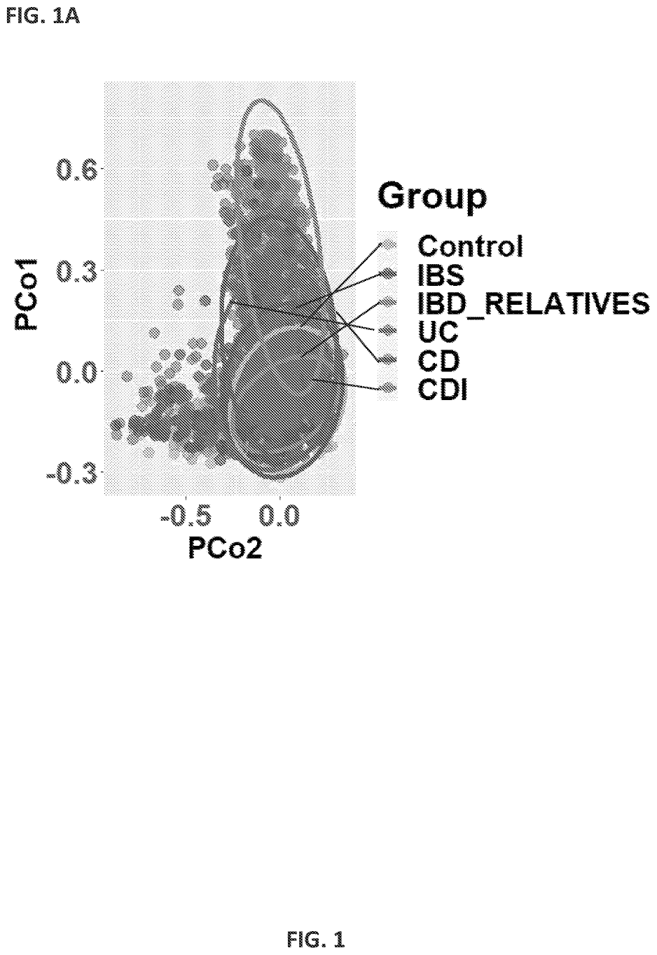

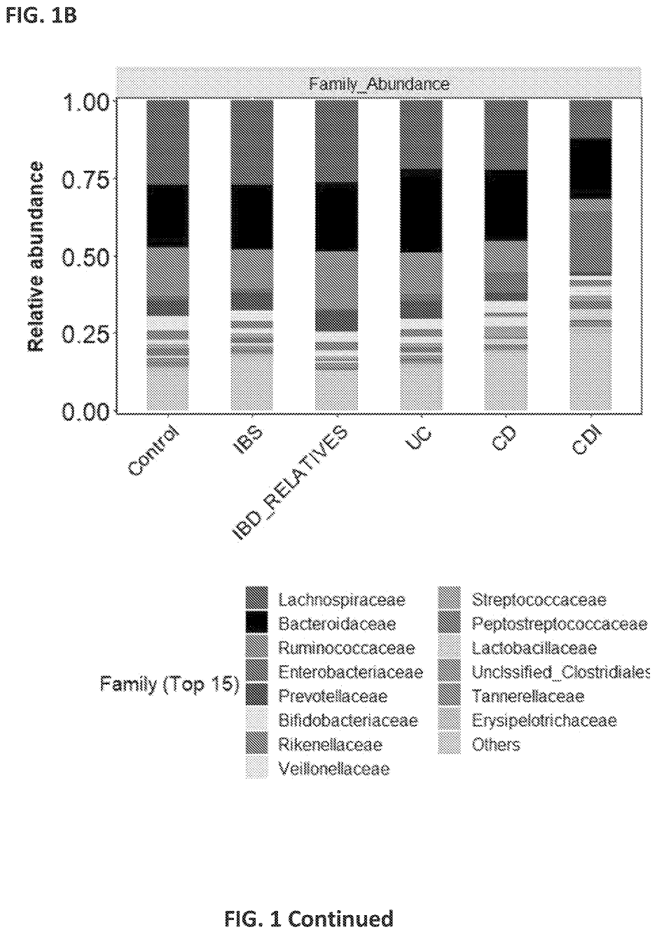

FIG. 1 shows gut microbiome structure and composition in diarrheal patients compared to controls. In FIG. 1A, a beta-diversity plot demonstrates that there are significant compositional shifts in disease groups, especially in patients with CDI and CD (ANOSIM p=0.001). A family abundance plot demonstrating compositional differences across disease groups are provided (FIG. 1B). Although there are some microbiome community differences associated with specific diseases, these do not clearly differentiate by beta-diversity or family abundance plots.

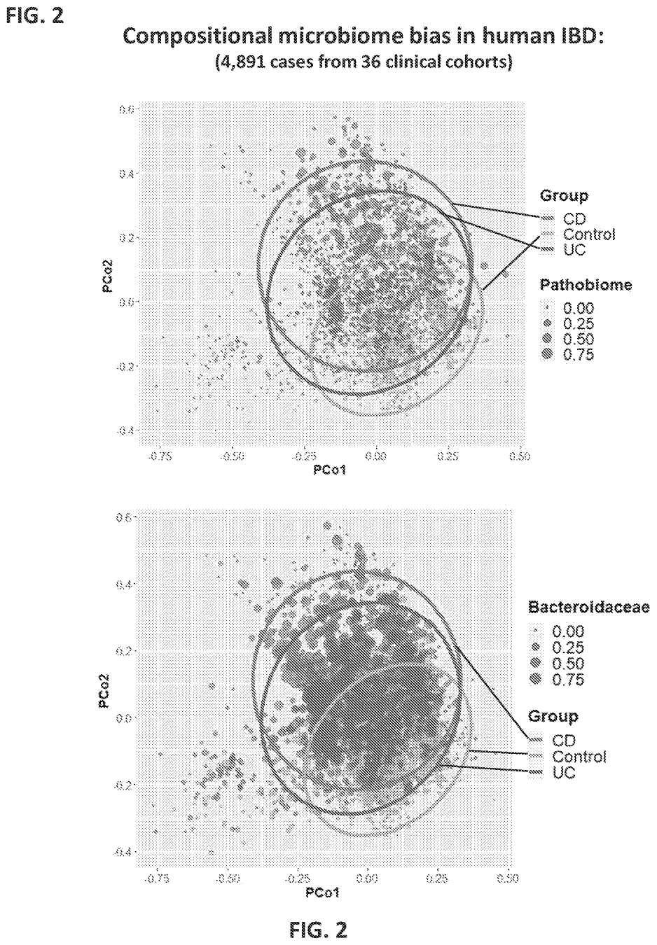

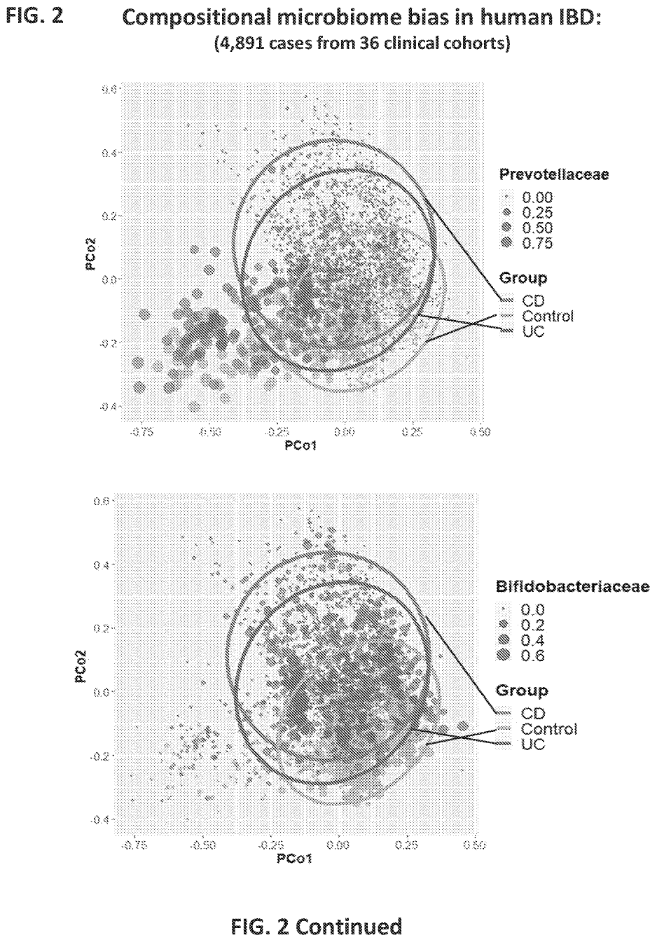

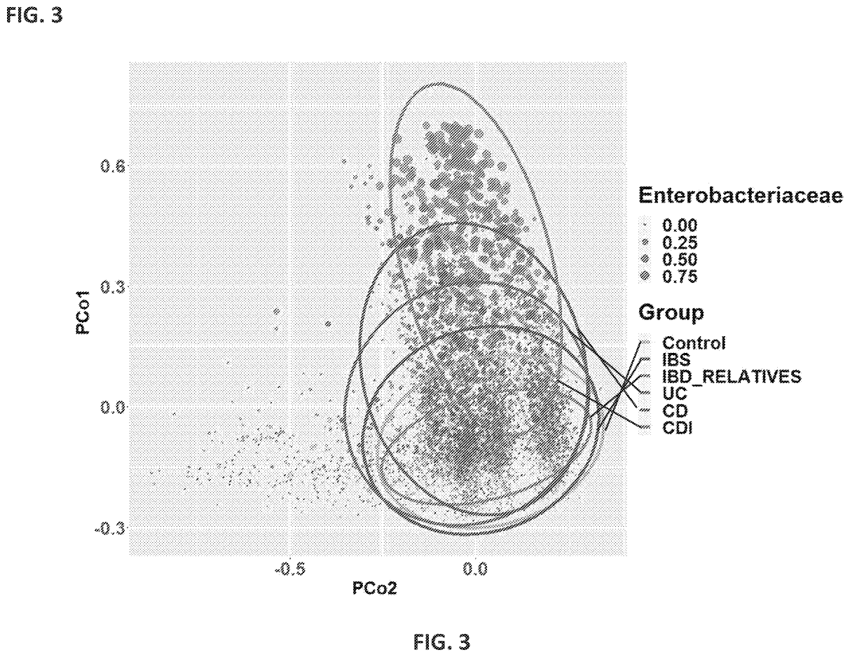

Compositional bias in human IBD is addressed in FIG. 2. The provided weighted Jaccard abundance distance plots demonstrate expansion of Pathobiome and Bacteroidaceae in CD and UC patients compared to controls, with loss at least of Bifidobacteriaceae. Certain microbiota features show a gradient-distribution in a beta-diversity plot, with enrichment of Pathobiome and Bacteroidaceae, and loss of Bifidobacteraceae in IBD patients. A beta-diversity plot demonstrating Enterbacteriaceae abundance is provided as an example of the Pathobiome expansion that occurs in human IBD and CDI patients (FIG. 3). Thus, microbiome community structure of IBD patients is different from their healthy relatives, who are similar with case-controls.

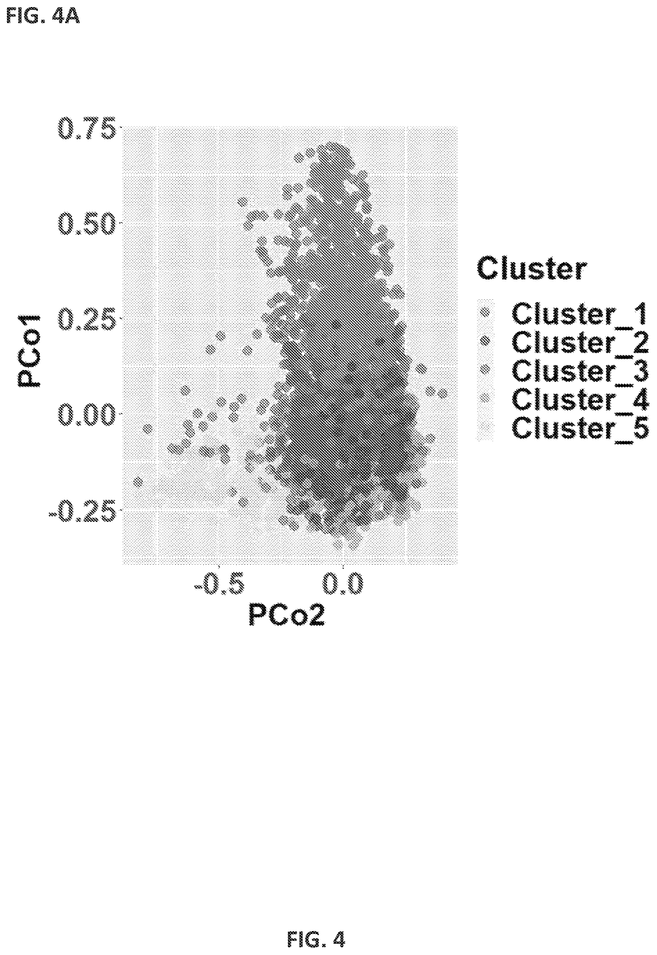

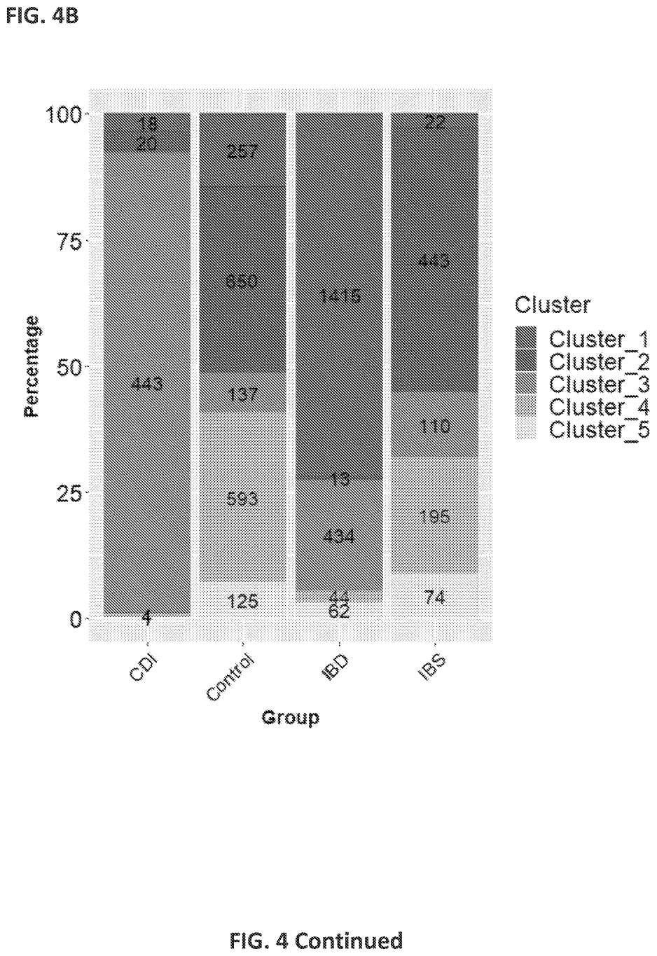

16S rRNA sequencing-based profiling of microbial community structure is illustrated by Dirichlet multinomial mixtures (DMM) clustering (FIG. 4) that is a probability-based model in which the samples are categorized based on the frequency of the appearance of each taxa in that sample. DMM identified 5 major clusters in an adult training 16S dataset (FIG. 4A), and at least some of the top drivers include as follows: Cluster 1 —Bacteroides, Blautia, Lachnospiraceae; Other; Cluster 2 —Bacteroides, Blautia, Faecalibacterium; Cluster 3 —Bacteroides, Blautia, Enterobacteriaceae; Other, Streptococcus; Cluster 4 —Blautia, Lachnospiraceae; Other, Faecalibacterium, Bifidobacterium and Cluster 5 —Prevotella. FIG. 4B provides a microbiome cluster representation in different human diarrheal diseases. Thus, IBD is characteristically dominated by cluster 1 and cluster 3 (cluster 1 being a relatively unique IBD cluster); whereas controls are dominated by clusters 2, 4 and 5. CDI patients are typically dominated by cluster 3; and IBS/FGID patients demonstrate a transition from cluster 4 (Bifidobacteria are significantly reduced) to cluster 2 and/or 3 enrichment (membership of Bacteroidaceae-dominant cluster 2 is expanded in IBS). Thus, disease-specific DMM clusters are distinct and apparent.

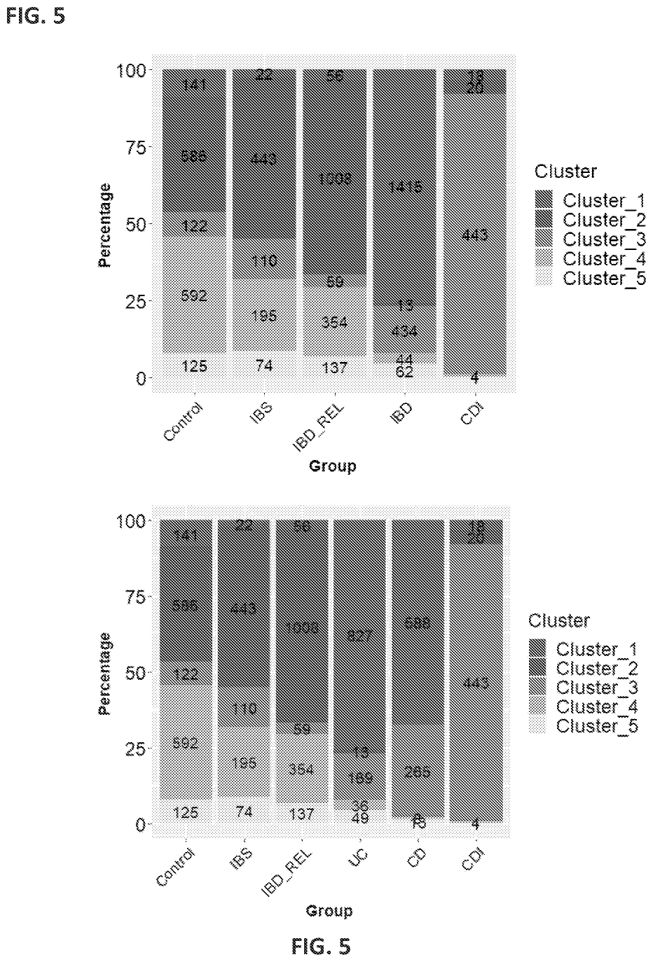

FIG. 5 shows that DMM clusters significantly differentiate different diarrheal disease specimens; Chi-squared analysis shows a significant cluster difference between disease groups in a combined analysis of 16S data using the Taxa4Meta profiler. Both IBD subtypes (UC and CD) show a unique disease cluster that is not evident in disease control patients (CDI or IBS). CDI patients are dominated by a cluster 3 microbiome composition.

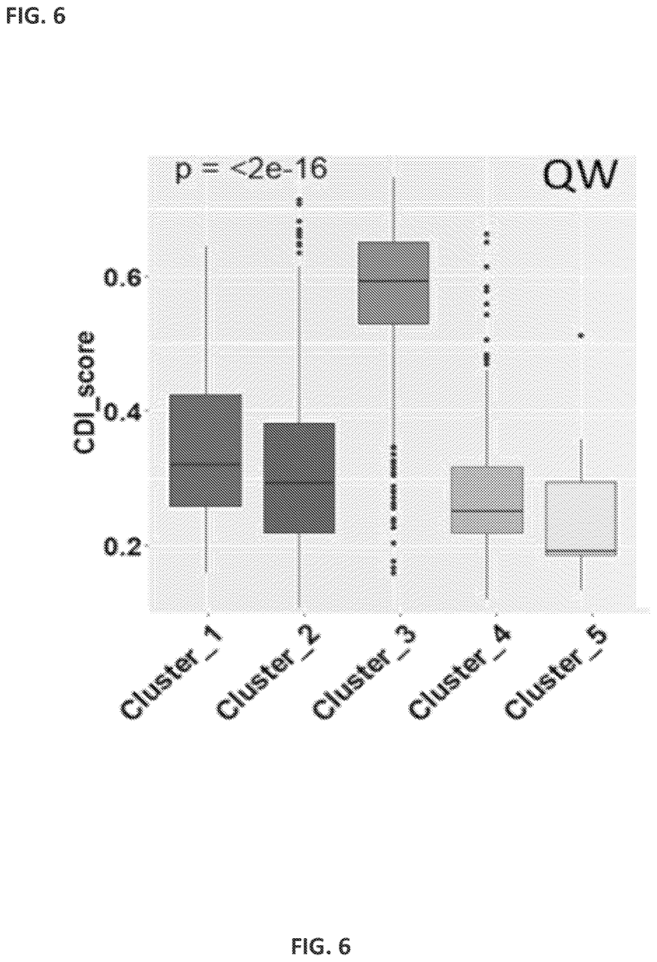

Microbiome features associated with CDI patients (i.e., a high CDI risk score) are significantly associated with dysbiotic cluster 3 (FIG. 6). In particular embodiments, a CDI diagnosis that is not classified as cluster 3 or a high CDI risk score should be re-tested for possible misdiagnosis. DMM classification differentiates CDI from IBS/IBD cases and provides an alternative diagnostic approach to establishing CDI/IBD/IBSrisk.

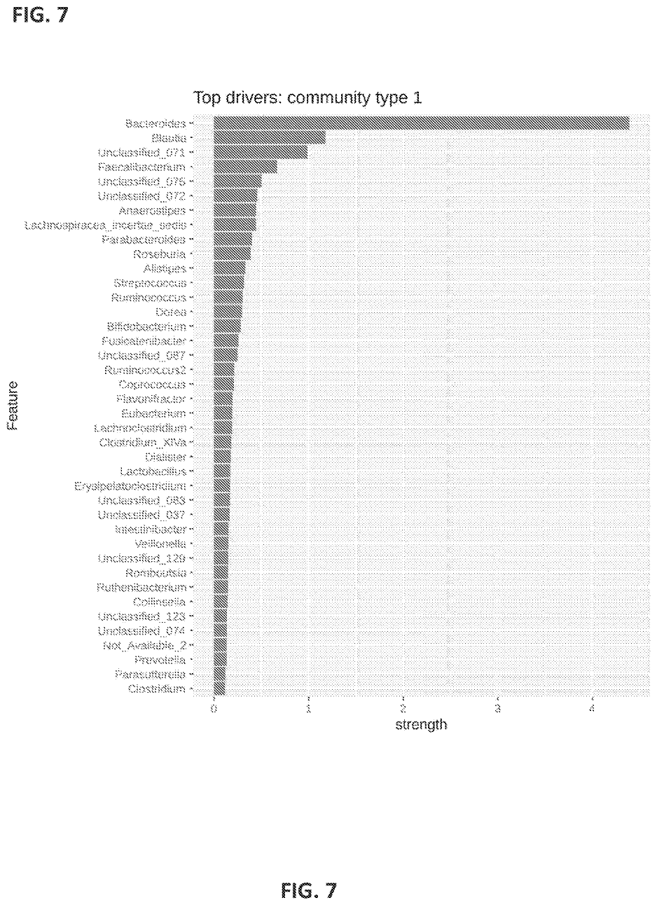

Top microbiome features that are associated with Cluster 1 are provided in FIG. 7. It is a Bacteroides-dominated cluster but it lacks protective species especially Faecalibacterium and Bifidobacterium. This cluster is significantly associated with IBD patients.

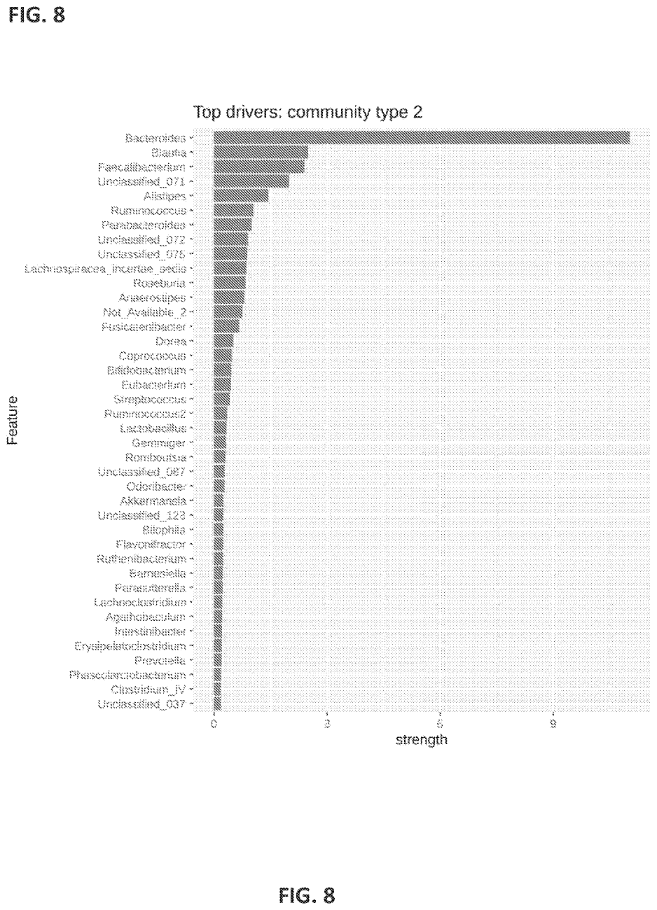

FIG. 8 demonstrates top microbiome features that are associated with Cluster 2. It is a Bacteroides-dominated cluster associated with healthy individuals, with higher abundance of taxa that are lacking in IBD patients, such as Faecalibacterium.

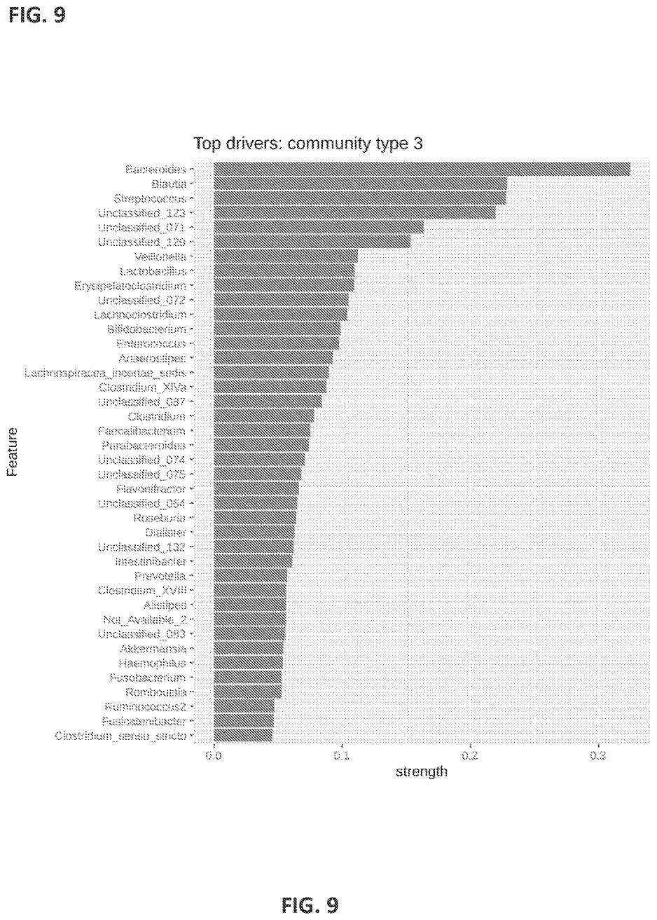

FIG. 9 shows top microbiome features that are associated with Cluster 3, which is a dysbiotic cluster enriched in pathobiome associated with CDI and some IBD patients.

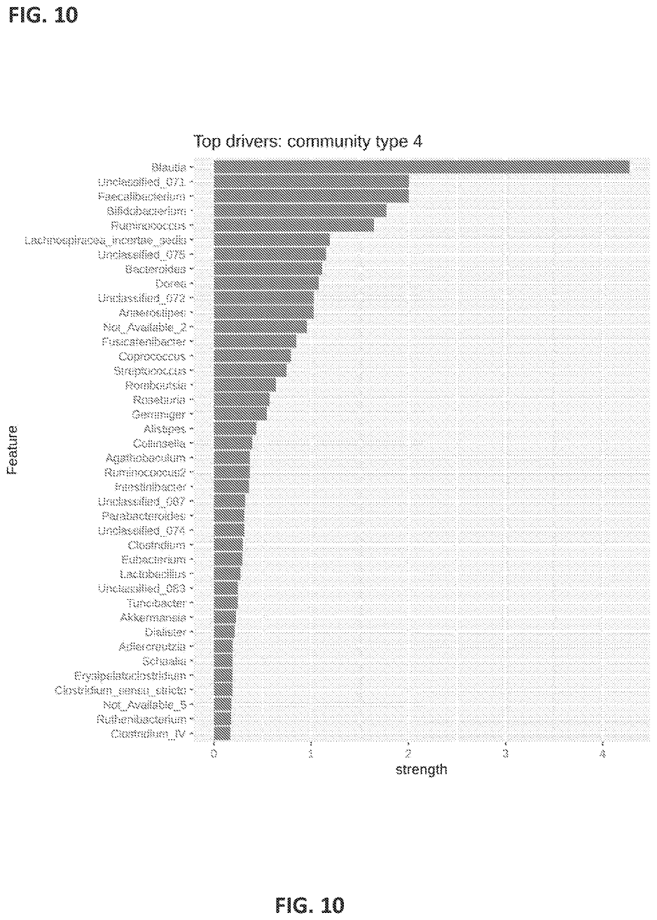

FIG. 10 shows top microbiome features that are associated with Cluster 4, which is a dominant Blautia cluster associated with healthy subjects that are highly enriched with protective taxa including Fecalibacteria, Bifidobacteria, and species such as E. rectale.

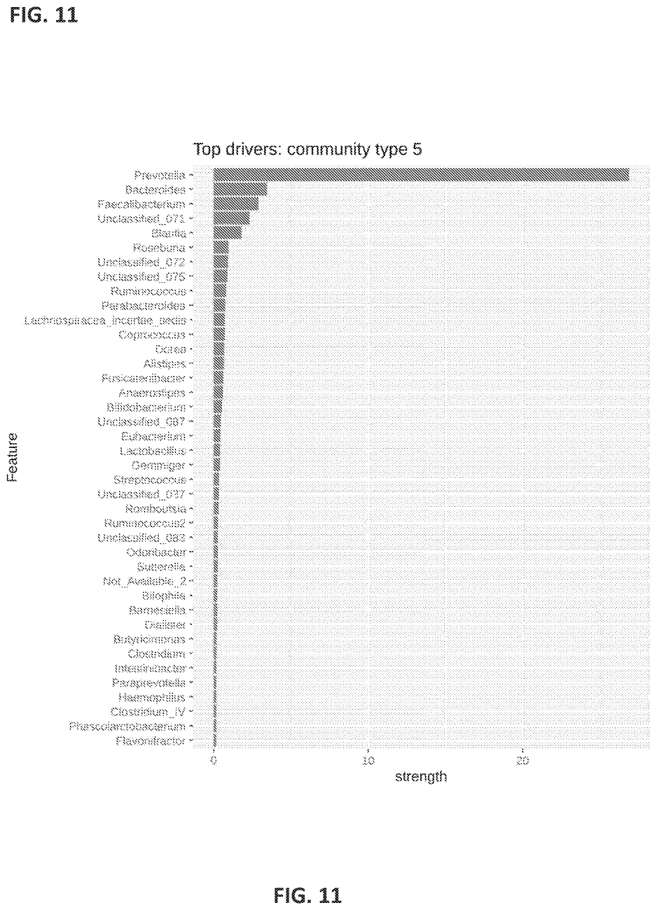

Top microbiome features that are associated with Cluster 5 are provided in FIG. 11, which is a Prevotella-dominated cluster associated with healthy individuals.

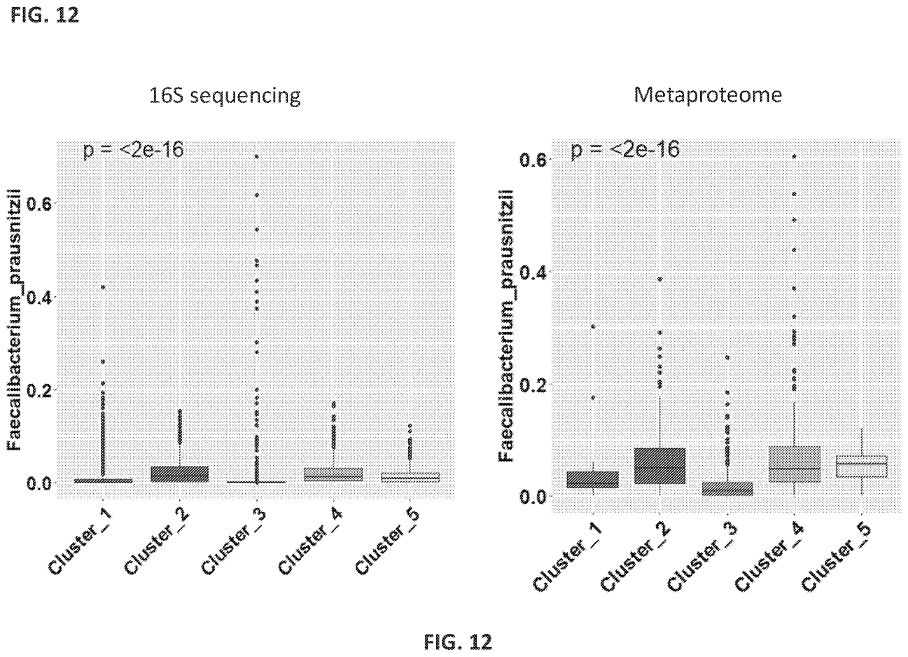

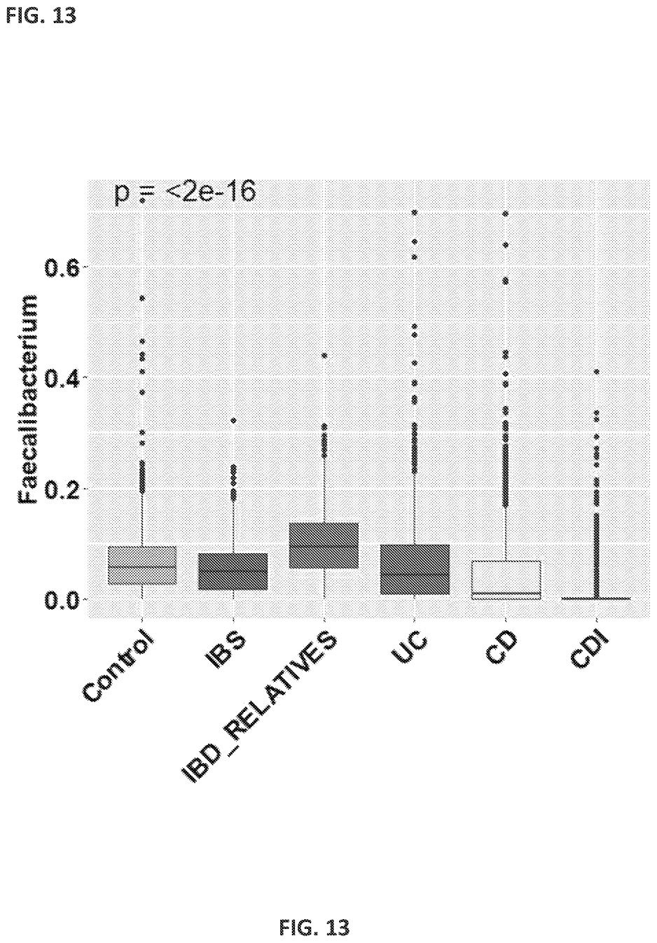

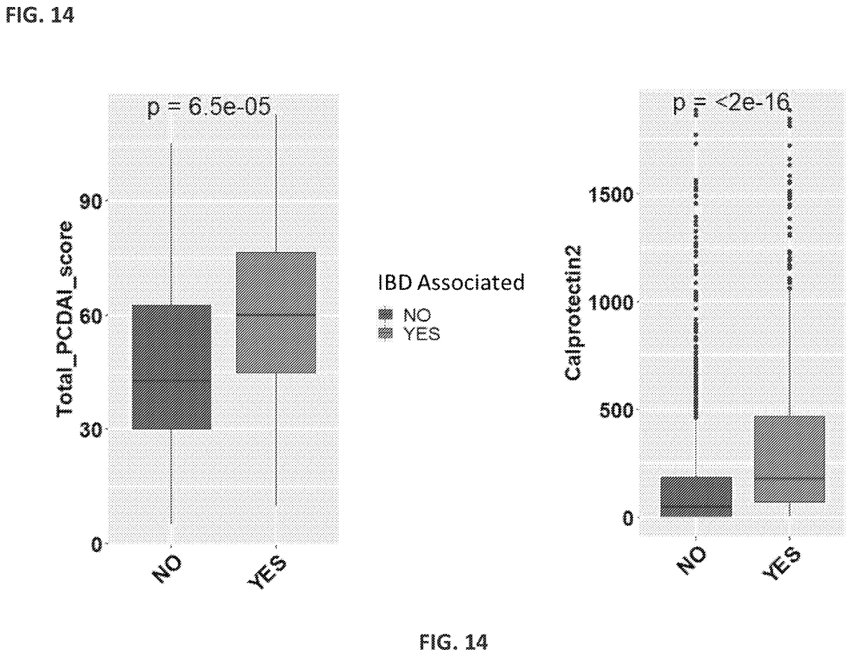

As demonstrated by demonstrated by 16S sequencing, FIG. 12 shows loss of Fecalibacteria in IBD-clusters 1 (and 3). Metaproteome analysis confirmed the cluster distribution of metabolically active Fecalibacteria in fecal samples of patients. FIG. 13 demonstrates loss of Fecalibacteria in IBD and CDI patients. Shotgun metaproteome analysis confirmed the disease associated clustering by demonstrating loss of metabolically active Fecalibacteria in fecal samples of IBD and CDI patients. Clinical symptoms and fecal calprotectin levels are illustrated in FIG. 14 and are elevated in subjects with an IBD cluster. Thus, in specific embodiments, IBD susceptibility is associated with Clusters 1 and 3 but not 2, 4, and 5.

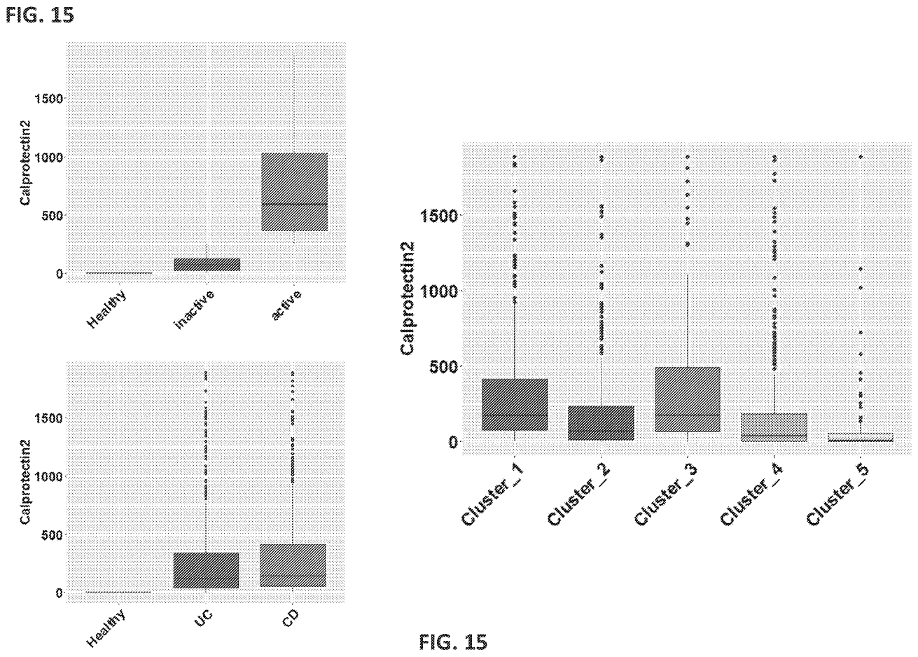

An independent cohort validation of fecal calprotectin 2 abundance (a marker of intestinal inflammation) by cluster and IBD subtype is provided in FIG. 15. Therein, IBD-clusters 1 and 3 are associated with an elevated fecal calprotectin. The IBD clusters are associated with more active intestinal inflammation.

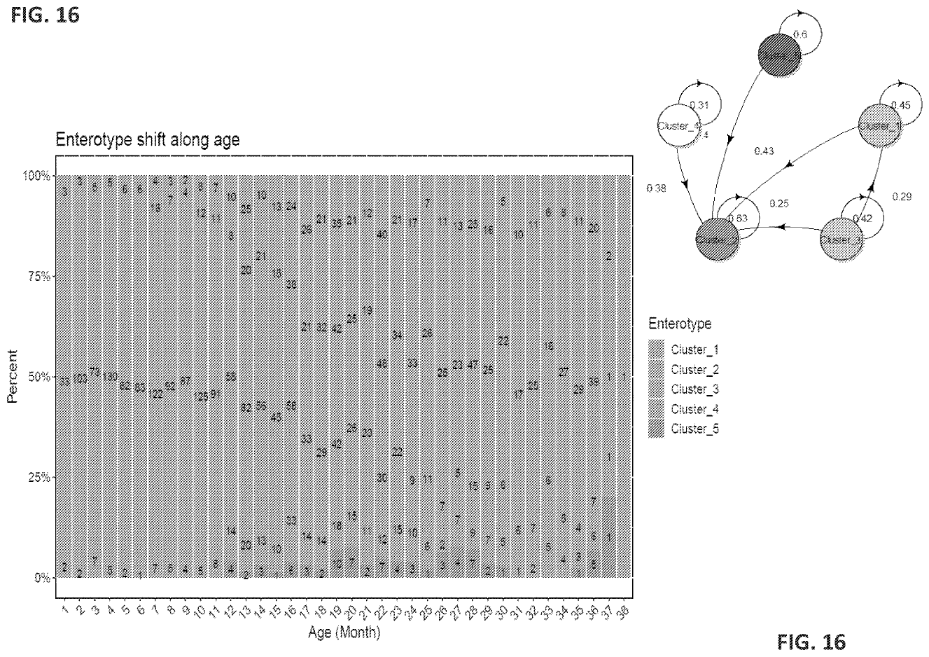

DIABIMMUNE is an early life human gut microbiome study that shows development of microbiome clusters modeled to the adult training set. Inter-enterotype transition probability at 1 and 3 years of age was analyzed and visualized using the Markov chain-based approach. Only transition probabilities greater than 0.2 are shown (FIG. 16; right). Healthy adult microbiome clusters 2, 4, and 5 start to emerge in infants after 12 months of age. Transitional probability analysis of the longitudinal development show that the IBD-associated clusters 1 and 3 represent immature transitional microbiome communities in infants.

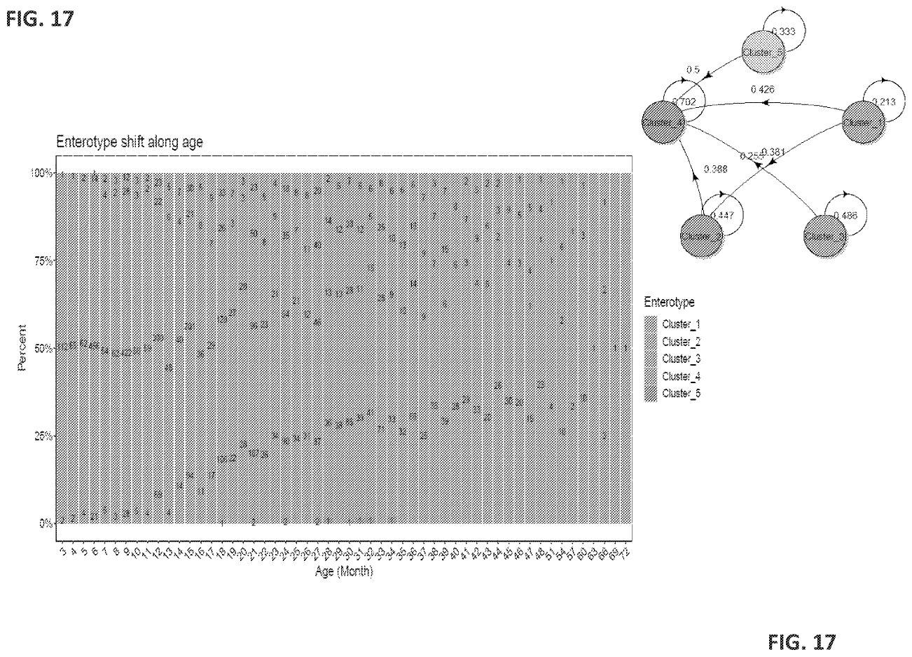

TEDDY is an early life human gut microbiome study that shows development of microbiome clusters modeled to the adult training set. Inter-enterotype transition probability at 1 and 3 years of age analyzed and visualized using the Markov chain-based approach. Only transition probabilities greater than 0.2 are shown (FIG. 17, right). Healthy adult microbiome clusters 2, 4 and 5 start to emerge in infants after 12 months of age. Transitional probability analysis of the longitudinal development show that the IBD-associated clusters 1 and 3 represent immature transitional microbiome communities in infants.

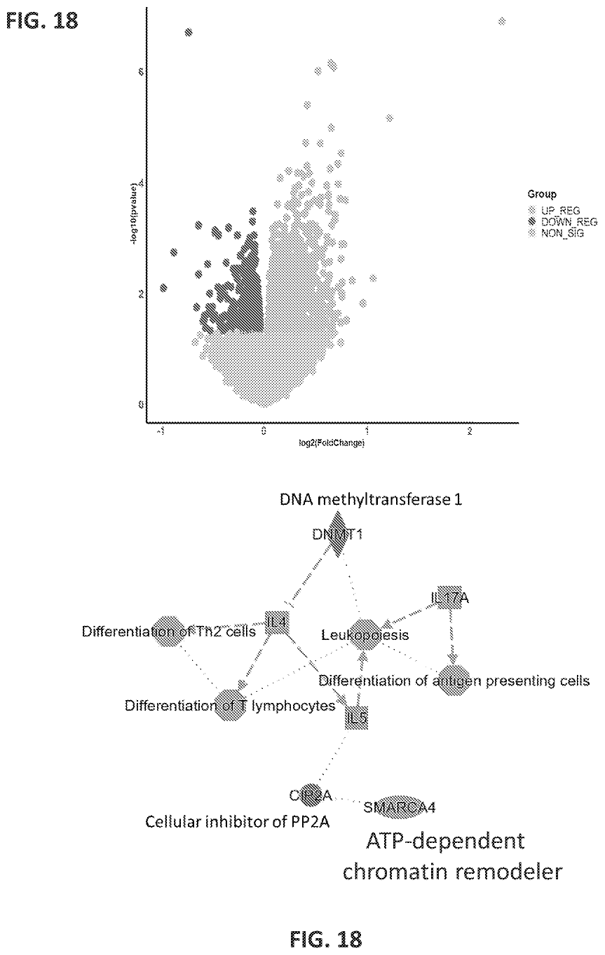

An RNASeq volcano plot (FIG. 18; top) of genes expressed in blood specimens from age and sex matched TEDDY infants with a healthy versus IBD microbiome cluster. (FIG. 18, bottom) Summary of RNA SEQ data shows that transition from the IBD to healthy-cluster is associated with significant development of T cell differentiation and immune tolerance, which is lacking in cluster 1. Therefore, infant and IBD microbiome composition shows a similar cluster composition which is associated with a lack of development of immune tolerance. In adults, IBD clusters are associated with microbiome immaturity and lack of immune development and tolerance. Methods to promote microbiome development and thereby immune maturation represents treatment for IBD patients, in specific embodiments.

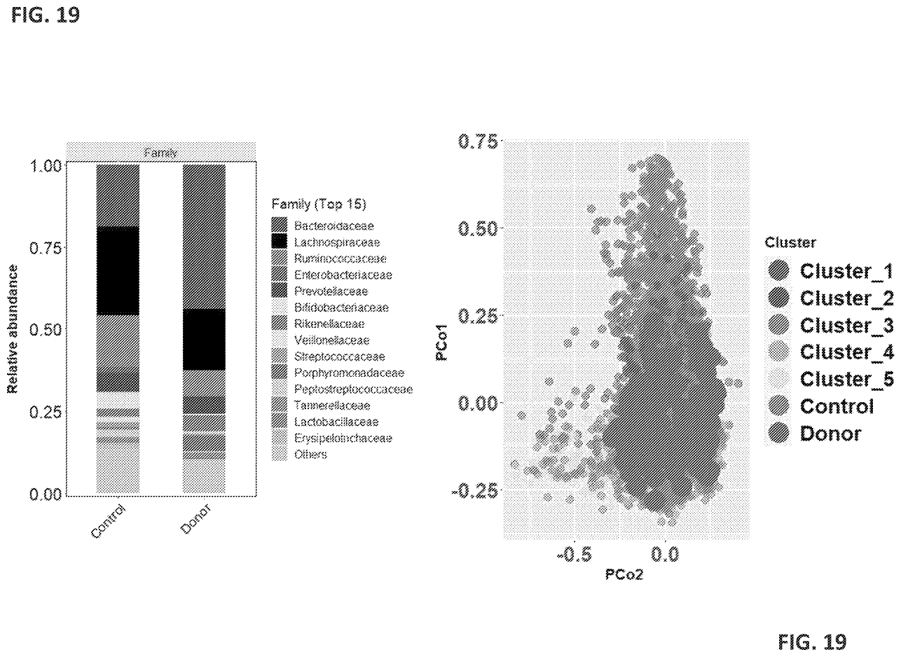

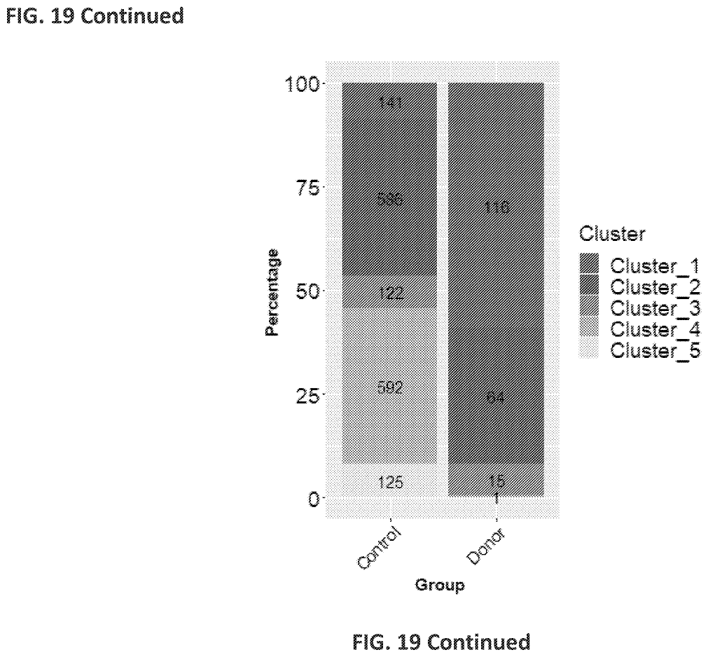

FMT considerations for treatment in IBD are provided in FIG. 19. FMT donor preparations reported in the literature demonstrate a microbiome community composition that is significantly different from healthy controls. Generally, FMT donor preparations lack key taxa present in the healthy gut microbiome Cluster 4, notably Fecalibacteria and Bifidobacteria (left). Donor preparations used to treat IBD patients are biased towards IBD risk clusters 1 and 3. Fecal donor preparations in specific embodiments lack dominant cluster 4 taxa that herein is demonstrated to be associated with IBD disease susceptibility and severity. It is demonstrated in animal IBD models that supplementation with these missing taxa is protective in IL10 deficient mice.

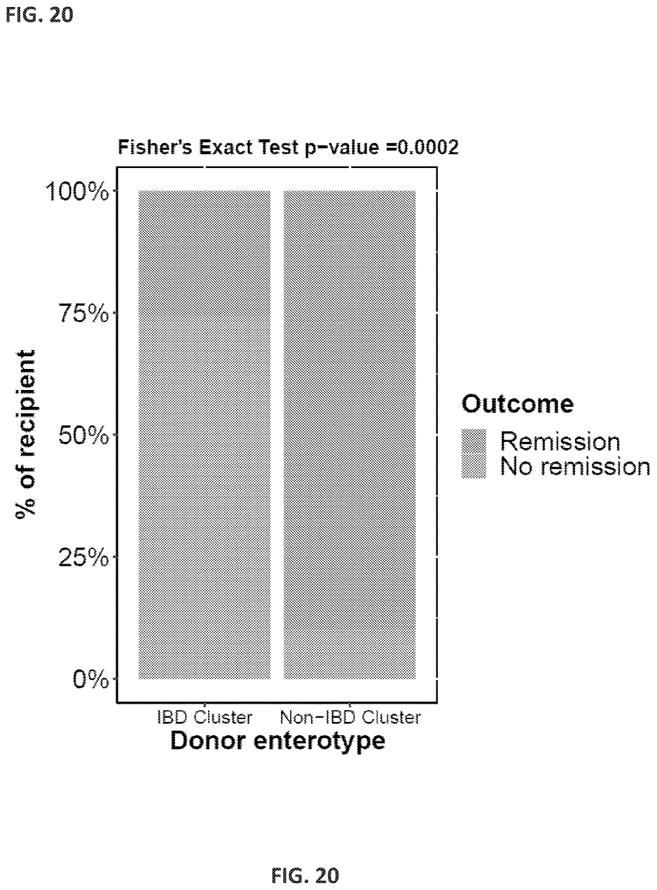

FIG. 20 shows clinical FMT outcomes in UC patients stratified by donor cluster. FMT treatment of UC patients with donor fecal preparations that comprise an IBD cluster are significantly more likely to experience clinical relapse. FMT donor preparations should be screened for therapeutic suitability.

TABLE 1
Unflassified Taxa Cluster details

| | Detail_taxonomy |
|---|---|
| Cluster_1 | |
| Unclassified_071 | Bacteria; Firmicutes; Clostridia; Clostridiales; Lachnospiraceae; Other |
| Unclassified_075 | Bacteria; Firmicutes; Clostridia; Clostridiales; Ruminococcaceae; Other |
| Unclassified_072 | Bacteria; Firmicutes; Clostridia; Clostridiales; Other; Other |
| Unclassified_087 | Bacteria; Other; Other; Other; Other; Other |
| Unclassified_037 | Bacteria; Bacteroidetes; Bacteroidia; Bacteroidales; Other; Other |
| Unclassified_083 | Bacteria; Firmicutes; Other; Other; Other; Other |
| Unclassified_129 | Bacteria; Proteobacteria; Gammaproteobacteria; Enterobacteriales; |
| | Enterobacteriaceae; Other |
| Unclassified_123 | Bacteria; Proteobacteria; Gammaproteobacteria; Enterobacterales; |
| | Enterobacteriaceae; Other |
| Unclassified_074 | Bacteria; Firmicutes; Clostridia; Clostridiales; Peptostreptococcaceae; Other |
| Not_Available_2 | Bacteria; Firmicutes; Clostridia; Clostridiales; Lachnospiraceae; Not Available |
| Cluster 2 | |
| Not_Available_2 | Bacteria; Firmicutes; Clostridia; Clostridiales; Lachnospiraceae; Not Available |
| Unclassified_037 | Bacteria; Bacteroidetes; Bacteroidia; Bacteroidales; Other; Other |
| Unclassified_071 | Bacteria; Firmicutes; Clostridia; Clostridiales; Lachnospiraceae; Other |
| Unclassified_072 | Bacteria; Firmicutes; Clostridia; Clostridiales; Other; Other |
| Unclassified_075 | Bacteria; Firmicutes; Clostridia; Clostridiales; Ruminococcaceae; Other |
| Unclassified 087 | Bacteria; Other; Other; Other; Other; Other |
| Unclassified_123 | Bacteria; Proteobacteria; Gammaproteobacteria; Enterobacterales; |
| | Enterobacteriaceae; Other |
| Cluster 3 | |
| Not_Available_2 | Bacteria; Firmicutes; Clostridia; Clostridiales; Lachnospiraceae; Not Available |
| Unclassified_071 | Bacteria; Firmicutes; Clostridia; Clostridiales; Lachnospiraceae; Other |
| Unclassified_072 | Bacteria; Firmicutes; Clostridia; Clostridiales; Other; Other |
| Unclassified_064 | Bacteria; Firmicutes; Bacilli; Lactobacillies; Other; Other |
| Unclassified_074 | Bacteria; Firmicutes; Clostridia; Clostridiales; Peptostreptococcaceae; Other |
| Unclassified_075 | Bacteria; Firmicutes; Clostridia; Clostridiales; Ruminococcaceae; Other |
| Unclassified_083 | Bacteria; Firmicutes; Other; Other; Other; Other |
| Unclassified_087 | Bacteria; Other; Other; Other; Other; Other |
| Unclassified_123 | Bacteria; Proteobacteria; Gammaproteobacteria; Enterobacterales; |
| | Enterobacteriaceae; Other |
| Unclassified_129 | Bacteria; Proteobacteria; Gammaproteobacteria; Enterobacteriales; |
| | Enterobacteriaceae; Other |
| Unclassified_132 | Bacteria; Proteobacteria; Gammaproteobacteria; Other; Other; Other |
| Cluster 4 | |
| Not_Available_2 | Bacteria; Firmicutes; Clostridia; Clostridiales; Lachnospiraceae; Not Available |
| Not_Available_5 | Bacteria; Firmicutes; Clostridia; Clostridiales; Ruminococcaceae; Not |
| | Available |
| Unclassified_071 | Bacteria; Firmicutes; Clostridia; Clostridiales; Lachnospiraceae; Other |
| Unclassified_072 | Bacteria; Firmicutes; Clostridia; Clostridiales; Other; Other |
| Unclassified_074 | Bacteria; Firmicutes; Clostridia; Clostridiales; Peptostreptococcaceae; Other |
| Unclassified_075 | Bacteria; Firmicutes; Clostridia; Clostridiales; Ruminococcaceae; Other |
| Unclassified_083 | Bacteria; Firmicutes; Other; Other; Other; Other |
| Unclassified_087 | Bacteria; Other; Other; Other; Other; Other |
| Cluster 5 | |
| Not_Available_2 | Bacteria; Firmicutes; Clostridia; Clostridiales; Lachnospiraceae; Not Available |
| Unclassified_037 | Bacteria; Bacteroidetes; Bacteroidia; Bacteroidales; Other; Other |
| Unclassified_071 | Bacteria; Firmicutes; Clostridia; Clostridiales; Lachnospiraceae; Other |
| Unclassified_072 | Bacteria; Firmicutes; Clostridia; Clostridiales; Other; Other |
| Unclassified_075 | Bacteria; Firmicutes; Clostridia; Clostridiales; Ruminococcaceae; Other |
| Unclassified_083 | Bacteria; Firmicutes; Other; Other; Other; Other |
| Unclassified_087 | Bacteria; Other; Other; Other; Other; Other |

### REFERENCES

All patents and publications mentioned in the specification are indicative of the level of those skilled in the art to which the inventions pertain. All patents and publications are herein incorporated by reference to the same extent as if each individual publication was specifically and individually indicated to be incorporated by reference.
Callahan, B. J. et al. DADA2: High-resolution sample inference from Illumina amplicon data. Nat. Methods 13, 581-583 (2016).
Cole, J. R. et al. Ribosomal Database Project: Data and tools for high throughput rRNA analysis. Nucleic Acids Res. 42, (2014).
Demšar, J. et al. Orange: Data mining toolbox in python. J. Mach. Learn. Res. 14, 2349-2353 (2013).
Duvallet, C., et al., Meta-analysis of gut microbiome studies identifies disease-specific and shared responses. Nat. Commun. 8, (2017).
Edgar, R. C. Search and clustering orders of magnitude faster than BLAST. Bioinformatics 26, 2460-2461 (2010).
Gao, X., et al., A Bayesian taxonomic classification method for 16S rRNA gene sequences with improved species-level accuracy. BMC Bioinformatics 18, (2017).
Hillmann, B. et al. Evaluating the Information Content of Shallow Shotgun Metagenomics. mSystems 3, (2018).
Mancabelli, L. et al. Identification of universal gut microbial biomarkers of common human intestinal diseases by meta-analysis. FEMS Microbiol. Ecol. 93, fix153 (2017).
Martin, M. Cutadapt removes adapter sequences from high-throughput sequencing reads. EMBnet.journal 17, 10 (2011).
McIntyre, A. B. R. et al. Comprehensive benchmarking and ensemble approaches for metagenomic classifiers. Genome Biol. 18, (2017).
Murali, A., Bhargava, A. & Wright, E. S. IDTAXA: A novel approach for accurate taxonomic classification of microbiome sequences. Microbiome 6, (2018).
Rognes, T., et al., F. VSEARCH: A versatile open source tool for metagenomics. Peer J 2016, (2016).
Saulnier, D. M. et al. Gastrointestinal microbiome signatures of pediatric patients with irritable bowel syndrome. Gastroenterology 141, 1782-1791 (2011).
Vich Vila, A. et al. Gut microbiota composition and functional changes in inflammatory bowel disease and irritable bowel syndrome. Sci. Transl. Med. 10, eaap8914 (2018).
Whon, T. W. et al. Data Descriptor: The effects of sequencing platforms on phylogenetic resolution in 16 S rRNA gene profiling of human feces. Sci. Data 5, (2018).

What is claimed is:

1. A method of treating and optionally diagnosing Inflammatory Bowel Disease (IBD) in a subject, comprising:
a) measuring taxa occurrence frequencies in at least one microbiome sample from a subject,
b) determining that the subject has or is at risk of developing IBD when the microbiome sample comprises enrichment in taxa Bacteroides, Blautia, and Lachnospiraceae, and depletion of taxa Faecalibacterium, Bifidobacteria, and Eubacteria, and
c) administering a therapy for the IBD.

2. The method of claim 1, wherein the therapy comprises fecal microbiota transplant (FMT), one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

3. The method of claim 1, wherein the therapy comprises FMT.

4. The method of claim 1, wherein measuring taxa occurrence frequencies in a subject microbiome further comprises classification of the subject microbiome into one of five Clusters, wherein each Cluster is associated with a taxa enrichment profile.

5. The method of claim 4, wherein:
a) Cluster 1 is enriched at least in, or only in, Bacteroides, Blautia, Lachnospiraceae, and unclassified taxa;
b) Cluster 2 is enriched at least in, or only in, Bacteroides, Blautia, and Faecalibacterium taxa;
c) Cluster 3 is enriched at least in, or only in, Bacteroides, Blautia, Enterobacteriaceae, Streptococcus, and unclassified taxa;
d) Cluster 4 is enriched at least in, or only in, Blautia, Lachnospiraceae, unclassified taxa, Faecalibacterium, Eubacteria, and Bifidobacterium; or
e) Cluster 5 is enriched at least in, or only in, Prevotella.

6. The method of claim 5, wherein Cluster 1 determines that the subject has or is at risk of developing IBD.

7. The method of claim 5, wherein the subject's microbiome is not enriched as in Cluster 2, Cluster 3, Cluster 4, and/or Cluster 5.

8. The method of claim 2, wherein the FMT is not enriched for taxa associated with Cluster 1 and/or Cluster 3.

9. The method of claim 1, wherein the loss of metabolically active Faecalibacterium, Bifidobacterium, and Eubacterium is associated with IBD and/or CDI.

10. The method of claim 1, wherein enrichment in calprotectin is associated with IBD and/or CDI.

11. The method of claim 1, wherein the microbiome sample or samples are grouped into one of five clusters.

12. The method of claim 1, wherein a dominant Blautia cluster is associated with healthy subjects.

13. The method of claim 2, wherein the FMT is enriched for taxa occurrences of Faecalibacterium, Eubacterium, and/or Bifidobacterium.

14. The method of any one of claims 5-13, wherein cluster 1 taxa profile comprises 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified_129, Romboutsia, Ruthenibacterium, Collinsella, Unclassified_123, Unclassified 074, Not_Available_2, Prevotella, Parasutterella, and Clostridium.

15. The method of claim 14, wherein cluster 1 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

16. The method of any one of claims 5-13, wherein cluster 2 taxa profile comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium_IV, and Unclassified_037.

17. The method of claim 16, wherein cluster 2 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

18. The method of any one of claims 5-13, wherein cluster 3 taxa profile comprises 1 or more of the following listed bacterial taxa: Bacteroides, Blautia, Streptococcus, Unclassified_123, Unclassified_071, Unclassified_129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified_075, Flavonifractor, Unclassified 064, Roseburia, Dialister, Unclassified 132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

19. The method of claim 18, wherein cluster 3 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

20. The method of any one of claims 5-13, wherein cluster 4 taxa profile comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified_074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV.

21. The method of claim 20, wherein cluster 4 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

22. The method of any one of claims 5-13, wherein cluster 5 taxa profile comprises: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available_2, Bilophila, Barnesiella, Dialister, Butyricimonas, Clostridium, Intestinibacter, Paraprevotella, Haemophilus, Clostridium IV, Phascolarctobacterium, and Flavonifractor.

23. The method of claim 22, wherein cluster 5 taxa profile comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

24. A method for selecting fecal samples suitable for fecal microbiota transplant (FMT), comprising:
analyzing at least one donor fecal sample microbiome to determine taxa Cluster enrichment, and selecting fecal samples that are not enriched in taxa Cluster 1 and/or Cluster 3 as suitable for FMT.

25. The method of claim 24, wherein a donor fecal sample is suitable for FMT when enriched in Cluster 2, Cluster 4, and/or Cluster 5.

26. The method of claim 24, wherein a donor fecal sample is suitable for FMT when enriched in Prevotella, Blautia, Faecalibacterium, Bifidobacteria, and/or Bacteroides.

27. The method of claim 24, wherein the donor microbiome fecal sample is selected at the time of fecal sample obtainment.

28. The method of claim 24, wherein the donor microbiome fecal sample is selected after fecal sample processing.

29. The method of claim 24, wherein the donor microbiome fecal sample is selected prior to administration of the FMT to a subject.

30. The method of claim 24, wherein the donor microbiome fecal sample is selected following administration of the FMT to a subject.

31. The method of claim 24, wherein the donor microbiome fecal sample is selected at least 1 week, 2 weeks, 3 weeks, 4 weeks, 2 months, 3 months, 4 months, 5 months, or 6 months after administration of the FMT to a subject.

32. The method of any one of claims 24-31, wherein cluster 2 taxa enrichment comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium IV, and Unclassified_037.

33. The method of claim 32, wherein cluster 2 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

34. The method of any one of claims 24-31, wherein cluster 4 taxa enrichment comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified_074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV.

35. The method of claim 38, wherein cluster 4 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

40. The method of any one of claims 24-31, wherein cluster 5 taxa enrichment comprises 1 or more of the following listed bacterial taxa: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available 2, Bilophila, Barnesiella, Dialister, Butyricimonas, Clostridium, Intestinibacter, Paraprevotella, Haemophilus, Clostridium IV, Phascolarctobacterium, and Flavonifractor.

36. The method of claim 40, wherein cluster 5 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

37. A kit, comprising:
(a) three or more bacteria species representative of at least three or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium IV, and Unclassified_037;
(b) three or more bacteria species representative of at least three or more of the following listed bacterial taxa: Blautia, Faecalibacterium, Bifidobacterium, Unclassified_071, Ruminococcus, Lachnospiraceae incertae sedis, Unclassified_075, Bacteroides, Dorea, Unclassified_072, Anaerostipes, Not_Available 2, Fusicatenibacter, Coprococcus, Streptococcus, Romboutsia, Roseburia, Gemmiger, Alistipes, Collinsella, Agathobaculum, Ruminococcus2, Intestinibacter, Unclassified 087, Parabacteroides, Unclassified_074, Clostridium, Eubacterium, Lactobacillus, Unclassified 083, Turicibacter, Akkermansia, Dialister, Adlercreutzia, Schaalia, Erysipelatoclostridium, Clostridium sensu stricto, Not_Available_5, Ruthenibacterium, and Clostridium IV; and/or
(c) three or more bacteria species representative of at least three or more of the following listed bacterial taxa: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available 2, Bilophila, Barnesiella, Dialister, Butyricimonas, Clostridium, Intestinibacter, Paraprevotella, Haemophilus, Clostridium IV, Phascolarctobacterium, and Flavonifractor.

38. The kit of claim 37, wherein the kit comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (a).

39. The kit of claim 37, wherein the kit comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (b).

40. The kit of claim 37, wherein the kit comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa in (c).

41. The kit of claim 37, wherein one or more of the bacteria in (a) are formulated for FMT.

42. The kit of claim 37, wherein one or more of the bacteria in (b) are formulated for FMT.

43. The kit of claim 37, wherein one or more of the bacteria in (c) are formulated for FMT.

44. The kit of claim 37, wherein the kit further comprises one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

45. The kit of claim 44, wherein the one or more anti-inflammatory drugs is an aminosalicylate.

46. The kit of claim 45, wherein the aminosalicylate is mesalamine, balsalazide, and/or olsalazine.

47. The kit of claim 37, wherein the one or more immune system suppressors is azathioprine, mercaptopurine and/or methotrexate.

48. The kit of claim 37, wherein the one or more small molecules is tofacitinib, upadacitinib, and/or ozanimod.

49. The kit of claim 37, wherein the one or more biologics is infliximab, adalimumab, golimumab, certolizumab, vedolizumab, ustekinumab, and/or Risankizumab.

50. The kit of claim 37, wherein the one or more antibiotics is ciprofloxacin, metronidazole, vancomycin, fidaxomicin, and/or bezlotoxumab.

51. The kit of claim 37, wherein the anti-diarrheal medication is psyllium powder, methylcellulose, and/or loperamide.

52. A method of diagnosing IBD in a subject, comprising:
a) measuring taxa occurrence frequencies in at least one microbiome sample from a subject,
b) determining that the subject has or is at risk of developing IBD when the microbiome sample comprises enrichment in taxa Bacteroides, Blautia, and Lachnospiraceae, and depletion of taxa Faecalibacterium, Bifidobacterium and Eubacterium.

53. The method of claim 52, wherein measuring taxa occurrence frequencies in a subject microbiome further comprises classification of the subject microbiome into one of five Clusters, wherein each Cluster is associated with a taxa enrichment profile.

54. The method of claim 53, wherein:
a) Cluster 1 is enriched at least in, or only in, Bacteroides, Blautia, Lachnospiraceae, and unclassified taxa;
b) Cluster 2 is enriched at least in, or only in, Bacteroides, Blautia, and Faecalibacterium taxa;
c) Cluster 3 is enriched at least in, or only in, Bacteroides, Blautia, Enterobacteriaceae, Streptococcus, and unclassified taxa;
d) Cluster 4 is enriched at least in, or only in, Blautia, Lachnospiraceae, unclassified taxa, Faecalibacterium, and Bifidobacterium; or
e) Cluster 5 is enriched at least in, or only in, Prevotella.

55. The method of claim 54, wherein Cluster 1 determines that the subject has or is at risk of developing IBD.

56. The method of claim 54, wherein the subject's microbiome is not enriched as in Cluster 2, Cluster 3, and/or Cluster 5.

57. The method of claim 54, wherein the loss of metabolically active Faecalibacterium, Bifidobacterium, and/or Eubacterium is associated with IBD and/or CDI.

58. The method of claim 54, wherein enrichment in calprotectin is associated with IBD and/or CDI.

59. The method of claim 54, wherein the microbiome sample or samples are grouped into one of five clusters.

60. The method of claim 54, wherein a dominant Blautia cluster is associated with healthy subjects.

61. The method of any one of claims 54-60, wherein cluster 1 taxa enrichment comprises 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified_129, Romboutsia, Ruthenibacterium, Collinsella, Unclassified_123, Unclassified 074, Not_Available_2, Prevotella, Parasutterella, and Clostridium.

62. The method of claim 61, wherein cluster 1 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

63. The method of any one of claims 54-60, wherein cluster 2 taxa enrichment comprises 1 or more of the following listed bacterial taxa: Bacteroides, Blautia, Faecalibacterium, Unclassified_071, Alistipes, Ruminococcus, Parabacteroides, Unclassified_072, Unclassified_075, Lachnospiraceae incertae sedis, Roseburia, Anaerostipes, Not_Available_2, Fusicatenibacter, Dorea, Coprococcus, Bifidobacterium, Eubacterium, Streptococcus, Ruminococcus2, Lactobacillus, Gemmiger, Romboutsia, Unclassified_087, Odoribacter, Akkermansia, Unclassified_123, Bilophila, Flavonifractor, Ruthenibacterium, Barnesiella, Parasutterella, Lachnoclostridium, Agathobaculum, Intestinibacter, Erysipelatoclostridium, Prevotella, Phascolarctobacterium, Clostridium IV, and Unclassified_037.

64. The method of claim 63, wherein cluster 2 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

65. The method of any one of claims 54-60, wherein cluster 3 taxa enrichment comprises 1 or more of the following listed bacterial taxa: Bacteroides, Blautia, Streptococcus, Unclassified_123, Unclassified_071, Unclassified 129, Veillonella, Lactobacillus, Erysipelatoclostridium, Unclassified 072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified 075, Flavonifractor, Unclassified 064, Roseburia, Dialister, Unclassified 132, Intestinibacter, Prevotella, Clostridium XVIII, Alistipes, Not_Available_2, Unclassified_083, Akkermansia, Haemophilus, Fusobacterium, Romboutsia, Ruminococcus2, Fusicatenibacter, and Clostridium sensu stricto.

66. The method of claim 65, wherein cluster 3 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

67. The method of any one of claims 54-60, wherein cluster 4 taxa enrichment comprises 1 or more of the following listed bacterial taxa: Blautia, Unclassified_071, Faecalibacterium, Bifidobacterium, Ruminococcus, Lachnospiraceae incertae sedis, Unclassified_075, Bacteroides, Dorea, Unclassified_072, Anaerostipes, Not_Available_2, Fusicatenibacter, Coprococcus, Streptococcus, Romboutsia, Roseburia, Gemmiger, Alistipes, Collinsella, Agathobaculum, Ruminococcus2, Intestinibacter, Unclassified_087, Parabacteroides, Unclassified_074, Clostridium, Eubacterium, Lactobacillus, Unclassified_083, Turicibacter, Akkermansia, Dialister, Adlercreutzia, Schaalia, Erysipelatoclostridium, Clostridium sensu stricto, Not_Available_5, Ruthenibacterium, and Clostridium IV.

68. The method of claim 67, wherein cluster 4 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

69. The method of any one of claims 54-60, wherein cluster 5 taxa enrichment comprises 1 or more of the following listed bacterial taxa: Prevotella, Bacteroides, Faecalibacterium, Unclassified_071, Blautia, Roseburia, Unclassified_072, Unclassified_075, Ruminococcus, Parabacteroides, Lachnospiraceae incertae sedis, Coprococcus, Dorea, Alistipes, Fusicatenibacter, Anaerostipes, Bifidobacterium, Unclassified_087, Eubacterium, Lactobacillus, Gemmiger, Streptococcus, Unclassified_037, Romboutsia, Ruminococcus2, Unclassified_083, Odoribacter, Sutterella, Not_Available 2, Bilophila, Barnesiella, Dialister, Butyricimonas, Clostridium, Intestinibacter, Paraprevotella, Haemophilus, Clostridium IV, Phascolarctobacterium, and Flavonifractor.

70. The method of claim 69, wherein cluster 5 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

71. The method of claim 54, further comprising administering one or more therapies to the subject.

72. The method of claim 54, further comprising administering one or more therapies to the subject identified as having a microbiome associated with Cluster 1 or Cluster 3.

73. The method of claim 71, wherein the therapy comprises FMT, one or more anti-inflammatory drugs, one or more corticosteroids, one or more immune system suppressors, one or more Biologics, one or more anti-diarrheal medications, one or more pain relievers, one or more vitamins and/or supplements, surgery, nutritional support, one or more small molecules, or a combination thereof.

74. The method of claim 73, wherein the FMT is not enriched for taxa associated with Cluster 1 and/or Cluster 3.

75. The method of claim 73, wherein the FMT is not enriched for taxa occurrences of Faecalibacterium and/or Bifidobacterium.

76. A method of diagnosing IBD in a subject, comprising:
a) measuring taxa occurrence frequencies in at least one microbiome sample from a subject suspected of having IBD or at risk for having IBD;
b) classifying the subject microbiome into one of five Clusters, wherein each Cluster is associated with a taxa enrichment profile, wherein a subject having a microbiome associated with Cluster 1 or Cluster 3 is determined to have IBD or be at risk for having IBD,
wherein
(I) Cluster 1 is enriched at least in, or only in, Bacteroides, Blautia, Lachnospiraceae, and unclassified taxa; or
Cluster 1 is enriched in 1 or more of the following listed bacterial taxa: Bacteroides, Blautia, Unclassified_071, Faecalibacterium, Unclassified_075, Unclassified_072, Anaerostipes, Lachnospiraceae incertae sedis, Parabacteroides, Roseburia, Alistipes, Streptococcus, Ruminococcus, Dorea, Bifidobacterium, Fusicatenibacter, Unclassified_087, Ruminococcus2, Coprococcus, Flavonifractor, Eubacterium, Lachnoclostridium, Clostridium XIVa, Dialister, Lactobacillus, Erysipelatoclostridium, Unclassified_083, Unclassified_037, Intestinibacter, Veillonella, Unclassified 129, Romboutsia, Ruthenibacterium, Collinsella, Unclassified_123, Unclassified 074, Not_Available_2, Prevotella, Parasutterella, and Clostridium; and
(II) Cluster 3 is enriched at least in, or only in, Bacteroides, Blautia, Enterobacteriaceae, Streptococcus, and unclassified taxa; or
Cluster 3 is enriched in 1 or more of the following listed bacterial taxa: Bacteroides, Blautia, Streptococcus, Unclassified_123, Unclassified_071, Unclassified_129, Veillonella, Lactobacillus, Erysipelatoclostridium, Unclassified_072, Lachnoclostridium, Bifidobacterium, Enterococcus, Anaerostipes, Lachnospiraceae incertae sedis, Clostridium XIVa, Unclassified_087, Clostridium, Faecalibacterium, Parabacteroides, Unclassified_074, Unclassified_075, Flavonifractor, Unclassified 064, Roseburia, Dialister, Unclassified_132, Intestinibacter, Prevotella, Clostridium XVIII, Alistipes, Not_Available 2, Unclassified_083, Akkermansia, Haemophilus, Fusobacterium, Romboutsia, Ruminococcus2, Fusicatenibacter, and Clostridium sensu stricto.

77. The method of claim 76, wherein cluster 1 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

78. The method of claim 76, wherein cluster 3 taxa enrichment comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of the listed bacterial taxa.

79. The method of claim 76, wherein the subject suspected of having IBD or at risk for having IBD has diarrhea or a family history of IBD.

80. The method of claim 76, wherein the subject suspected of having IBD or at risk for having IBD has a microbiome that comprises enrichment in taxa Bacteroides, Blautia, and/or Lachnospiraceae and/or has depletion of taxa Faecalibacterium, Bifidobacteria, and/or Eubacteria.

81. The method of claim 79, wherein the diarrhea is chronic.

82. A method of diagnostic surveillance and prophylactic management of Inflammatory Bowel Disease (IBD) risk in a subject, comprising:
a) measuring taxa occurrence frequencies in at least one microbiome sample from a subject,
b) determining that the subject has or is at risk of developing IBD when the microbiome sample comprises enrichment in taxa Bacteroides, Blautia, and Lachnospiraceae, and depletion of taxa Faecalibacterium, Bifidobacteria, and Eubacteria and
c) administering a therapy for the IBD.

83. The method of claim 82, wherein the subject has no diarrhea.

84. The method of claim 82, wherein the subject has diarrhea.

85. The method of claim 82, wherein the individual is healthy.

86. The method of claim 82, wherein the subject has a biological family member with IBD.

87. The method of claim 82, wherein the method is performed periodically.

88. The method of claim 82, wherein the method is performed annually.

89. The method of claim 82, wherein the subject has one or more IBD-gene linkage susceptibility loci.

90. The method of claim 82, wherein the subject has a microbiome Cluster 1 or Cluster 3 and/or has one or more IBD-gene linkage susceptibility loci.

91. The method of claim 90, wherein the subject is provided an effective amount of prophylactic management of disease risk.

92. The method or kit of any one of the preceding claims, wherein one or more Unclassified and/or Not Available taxa are removed from clusters 1, 2, 3, 4, and/or 5.
